(19) United States
(12) Patent Application Publication
(10) Pub. No.: US 2020/0009105 A1
(43) Pub. Date: Jan. 9, 2020

(54) PROSTACYCLIN, PROSTACYCLIN ANALOGS, AND METHODS OF TREATING OR PREVENTING REJECTION OF SOLID ORGAN TRANSPLANTS

(71) Applicants: VANDERBILT UNIVERSITY, Nashville, TN (US); THE UNITED STATES AS REPRESENTED BY THE DEPARTMENT OF VETERANS AFFAIRS, Washington, DC (US)

(72) Inventor: Stokes PEEBLES, Nashville, TN (US)

(73) Assignees: Vanderbilt University, Nashville, TN (US); The United States as Represented by The Departmet of Veterans Affairs, Washington, DC (US)

(21) Appl. No.: 16/489,382

(22) PCT Filed: Feb. 28, 2018
(86) PCT No.: PCT/US2018/020109

§ 371 Date: Aug. 28, 2019

Related U.S. Application Data
(60) Provisional application No. 62/464,580, filed on Feb. 28, 2017.

(51) Int. Cl. A61K 31/343; A61K 31/191; A61P 11/08; A61P 37/06

(52) U.S. Cl. CPC A61K 31/343; A61P 37/06; A61P 11/08; A61K 31/191

(57) ABSTRACT

The present disclosure relates to methods for treating or preventing rejection of solid organ transplants by administering prostacyclin (PGI₂) or a prostacyclin analog.

### CROSS REFERENCE TO RELATED APPLICATIONS

This application claims the benefit of U.S. Provisional Patent Application Ser. No. 62/464,580 filed Feb. 28, 2017, which is expressly incorporated herein by reference.

### STATEMENT REGARDING FEDERALLY SPONSORED RESEARCH

This invention was made with Government Support under Grant Nos. R01 HL 090664, U19 AI 95227, R01 AI 111820, T32 GM 007347, F30 AI118376-01, R56 AI076411 awarded by the National Institutes of Health and Grant No. 2I01BX 000624 awarded by the Department of Veterans Affairs. The Government has certain rights in the invention.

### FIELD

The present disclosure relates to methods for treating or preventing rejection of solid organ transplants by administering prostacyclin (PGI₂) or a prostacyclin analog.

### BACKGROUND

Solid organ transplantation is a life-changing therapy for end stage diseases of the heart, kidney, lung, liver, and pancreas. Major barriers to long-term transplant graft survival are acute and chronic rejection, processes which are largely mediated by T and B lymphocytes that cause graft dysfunction. Immunosuppressive therapy is a major strategy to decrease T cell-mediated rejection; however, there are significant side effects that complicate their use.

Calcineurin inhibitors are a widely used component of current immunosuppressive regimens to prevent T lymphocyte-mediated rejection. Calcineurin inhibitors, such as cyclosporine and tacrolimus prevent activation of nuclear factor of activated T cells, cytoplasmic 1 (NFATc) and thus inhibit transcription of the IL2 gene, resulting in the beneficial immunosuppressive effects of this class of medications to prevent transplant rejection.

However, calcineurin inhibitors have significant adverse effects such as hypertension and renal dysfunction that may limit their use, thus increasing the risk of graft rejection. Thus, a pharmacologic agent that inhibits NFATc activation, yet that does not have the deleterious side effects that occur with the use of calcineurin inhibitors, would be a major therapeutic advance in preventing solid organ graft rejection.

The compositions and methods disclosed herein address these and other needs.

### SUMMARY

Disclosed herein are methods for treating or preventing rejection of solid organ transplants comprising administering prostacyclin (PGI₂) or a prostacyclin analog. The inventor has surprisingly discovered that prostacyclin analogs significantly down-regulate IL-2 production, CD25 expression, IL-2R signaling, and NFATc activation. PGI₂ and analogs thereof have many of the immunoinhibitory features of calcineurin inhibitors without the toxicity associated with those drugs. This technology advances the field of transplantation medicine, and provides a paradigm shift in identifying a novel therapeutic strategy to treat solid organ transplant recipients to decrease rejection of the incredibly valuable resource of human organs.

In some aspects, disclosed herein is a method of treating or preventing solid organ transplant rejection comprising administering to a subject in need thereof prostacyclin or a prostacyclin analog, or a pharmaceutically acceptable salt thereof.

In some embodiments, the subject is administered prostacyclin. In some embodiments, the prostacyclin is epoprostenol.

In some embodiments, the subject is administered a prostacyclin analog. In some embodiments, the prostacyclin analog is selected from iloprost, cicaprost, treprostinil, or beraprost. In some embodiments, the prostacyclin analog is iloprost.

In some embodiments, the prostacyclin or a prostacyclin analog is administered by a method selected from the group consisting of intravenous, oral, and aerosol.

In some embodiments, the solid organ transplant is a heart, kidney, lung, liver, or pancreas transplant. In some embodiments, the solid organ transplant is a heart transplant. In some embodiments, the solid organ transplant is a kidney transplant.

### BRIEF DESCRIPTION OF THE DRAWINGS

The accompanying figures, which are incorporated in and constitute a part of this specification, illustrate several aspects described below.

FIG. 1. Cicaprost decreased IL-33-induced IL-4, IL-5, IL-13 expression by CD4 T cells. Naïve CD4 cells from (A-C) BALB/c, IP KO, or (D and E) WT mice were stimulated with anti-CD3 and anti-CD28 Abs with or without IL-33 and were treated with cicaprost or vehicle for 3 days. A-C. The levels of IL-4, IL-5, and IL-13 in the culture supernatant were determined by ELISA. D and E. IL-4 and IL-13 expressing cells were determined by flow cytometry. Data are combined of 3 experiments and presented as mean±SEM. *, p<0.05, n=9.

FIG. 2. Cicaprost decreased Gata3 expression in CD4 T cells. Naïve CD4 cells from BALB/c or IP KO mice were stimulated with anti-CD3 and anti-CD28 Abs with or without IL-33 and were treated with cicaprost or vehicle for 3 days. The levels of Gata3 protein expression in the cells was determined by flow cytometry. A. Flow plots. B. Gata3 mean fluorescent intensity (MFI). C. the percentages of Gata3⁺ cells. D. Total Gata3⁺ cells. Data are combined of 3 experiments and presented as mean±SEM. *, p<0.05, n=9.

FIG. 3. Cicaprost decreased IL-2 production and NFAT activation in CD4 T cells. Naïve CD4 cells from BALB/c or IP KO mice were stimulated with anti-CD3 and anti-CD28 Abs with or without IL-33 and were treated with cicaprost or vehicle. A. The levels of IL-2 in the culture supernatant at day 2 were determined by ELISA. B. Cells were transduced with NFAT-luciferase reporter lentiviral vector or vehicle vector. Cells were harvested on day 3 for luciferase assay. Data are combined of 3 (A) and 2 (B) experiments and presented as mean±SEM. *, p<0.05, n=9 (A) and 6 (B).

FIG. 4. Cicaprost inhibited CD4 T cell proliferation. Naïve CD4 cells from BALB/c or IP KO mice were stimulated with anti-CD3 and anti-CD28 Abs with or without IL-33 and were treated with cicaprost or vehicle. A. CFSE-labeled cells were cultured and treated for 3 days and cell division was analyzed by flow cytometry. B. Cell division index. C. Total numbers of live cells. Data are combined of 5 (B and C) experiments and presented as mean±SEM. *, p<0.05, n=9 (A) and 15 (C-D).

FIG. 5. Cicaprost decreased type 2 cytokine production by CD4 T cells in the presence of exogenous IL-2. Naïve CD4 T cells from BALB/c or IP KO mice were stimulated with anti-CD3 and anti-CD28 Abs with or without IL-33 and IL-2 and were treated with cicaprost or vehicle for 3 days. A-B. The levels of IL-5 (A) and IL-13 (B) in the culture supernatant were determined by ELISA. Data are combined of 4 experiments and presented as mean±SEM. *, p<0.05, n=8-11.

FIG. 6. Cicaprost decreased CD25 expression and IL-2 receptor signaling in CD4 T cells. Naïve CD4 cells from BALB/c or IP KO mice were stimulated with anti-CD3 and anti-CD28 Abs with or without IL-33 and were treated with cicaprost or vehicle for 3 days (A-D) or 2 days (E). A-D. The levels of CD25 protein expression on the cell surface were determined by flow cytometry. A. Flow gating strategy. B. CD25 mean fluorescent intensity (MFI). C. The percentages of CD25⁺CD4⁺ T cells. C. Total numbers of CD25⁺CD4⁺ T cells. E. The levels of p-STAT5 in the cell lysate determined by ELISA. Data are combined of 3 experiments and presented as mean±SEM. *, p<0.05, n=9.

FIG. 7. Gating strategy for isolation of Treg by Foxp3 expression. Splenic T cells were isolated from Foxp3 GFP+ transgenic mice that express green fluorescent protein in Foxp3+ Treg.

FIG. 8. IP signaling increases per cell Foxp3 expression. Spleen cells were isolated from Foxp3-GFP and Foxp3-GFP x IP KO mice and Tregs were defined as CD4+CD25+Foxp3+ using the flow gating strategy shown in FIG. 4. Treg from WT mice had significantly greater Foxp3 expression based on MFI than IP KO mice. ****p<0.0001

FIG. 9. Treg from IP knockout mice are less suppressive than WT Tregs in vitro. WT Teff cells were activated with anti-CD3 and anti-CD28 in the presence of Treg from either Foxp3-GFP (red lines) or Foxp3-GFP x IP KO mice (blue lines) at ratios of 1:4, 1:2, or 1:1. Tregs from IP KO mice had significantly less suppressive function than Treg from WT mice at a ratio of 1 Treg:1 Teff. *p<0.05.

FIG. 10. Treg from IP knockout mice produced less IL-10 than WT Tregs in vitro. Foxp3-GFP expressing Treg from WT and IP KO mice were activated with anti-CD3 and anti-CD28 and culture supernatant was collected daily for IL-10 measurement. Tregs from IP KO mice had significantly decreased IL-10 secretion at day 3 compared to WT Treg. *p<0.05.

FIG. 11. Endogenous PGI₂ signaling inhibits lung IL-13 following 4 consecutive days of IT Alt Ex.

FIG. 12. Endogenous PGI₂ signaling reduces the number of lung ILC2 following 4 consecutive days of IT Alt Ex.

### DETAILED DESCRIPTION

Disclosed herein are methods for treating or preventing rejection of solid organ transplants comprising administering prostacyclin (PGI₂) or a prostacyclin analog. The inventor has surprisingly discovered that prostacyclin analogs significantly down-regulate IL-2 production, CD25 expression, IL-2R signaling, and NFATc activation.

PGI₂ and analogs thereof have many of the immunoinhibitory features of calcineurin inhibitors without the toxicity associated with those drugs. This technology advances the field of transplantation medicine, and provides a paradigm shift in identifying a novel therapeutic strategy to treat solid organ transplant recipients to decrease rejection of the incredibly valuable resource of human organs.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed.

The following definitions are provided for the full understanding of terms used in this specification.

### Terminology

As used in the specification and claims, the singular form “a,” “an,” and “the” include plural references unless the context clearly dictates otherwise. For example, the term “a cell” includes a plurality of cells, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, a “therapeutically effective amount” of a therapeutic agent refers to an amount that is effective to achieve a desired therapeutic result, and a “prophylactically effective amount” of a therapeutic agent refers to an amount that is effective to prevent an unwanted physiological condition. Therapeutically effective and prophylactically effective amounts of a given therapeutic agent will typically vary with respect to factors such as the type and severity of the disorder or disease being treated and the age, gender, and weight of the subject.

The term “therapeutically effective amount” can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

As used herein, the term “mixture” can include solutions in which the components of the mixture are completely miscible, as well as suspensions and emulsions, in which the components of the mixture are not completely miscible.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

### Methods

In some aspects, disclosed herein is a method of treating or preventing solid organ transplant rejection comprising administering to a subject in need thereof prostacyclin or a prostacyclin analog, or a pharmaceutically acceptable salt thereof.

In some embodiments, the subject is administered prostacyclin. In some embodiments, the prostacyclin is epoprostenol.

In some embodiments, the subject is administered a prostacyclin analog. In some embodiments, the prostacyclin analog is selected from iloprost, cicaprost, treprostinil, or beraprost. In some embodiments, the prostacyclin analog is iloprost. In some embodiments, the prostacyclin analog is cicaprost. In some embodiments, the prostacyclin analog is treprostinil. In some embodiments, the prostacyclin analog is beraprost.

In some embodiments, the prostacyclin or a prostacyclin analog is administered by a method selected from the group consisting of intravenous, oral, and aerosol.

In some embodiments, the administration of the prostacyclin or the prostacyclin analog decreases IL33 expression (RNA or protein). In some embodiments, the administration of the prostacyclin or the prostacyclin analog decreases IL2 expression (RNA or protein). In some embodiments, the administration of the prostacyclin or the prostacyclin analog decreases CD25 expression (RNA or protein). In some embodiments, the administration of the prostacyclin or the prostacyclin analog decreases IL-2R expression (RNA or protein). In some embodiments, the administration of the prostacyclin or the prostacyclin analog decreases Gata3 expression (RNA or protein).

In some embodiments, the solid organ transplant is a heart, kidney, lung, liver, or pancreas transplant. In some embodiments, the solid organ transplant is a heart transplant. In some embodiments, the solid organ transplant is a kidney transplant. In some embodiments, the solid organ transplant is a lung transplant. In some embodiments, the solid organ transplant is a liver transplant. In some embodiments, the solid organ transplant is a pancreas transplant.

In some additional aspects, disclosed herein is a method of treating or preventing allergic disease comprising administering to a subject in need thereof prostacyclin or a prostacyclin analog, or a pharmaceutically acceptable salt thereof.

In some embodiments, the subject is administered prostacyclin. In some embodiments, the prostacyclin is epoprostenol.

In some embodiments, the subject is administered a prostacyclin analog. In some embodiments, the prostacyclin analog is selected from iloprost, cicaprost, treprostinil, or beraprost. In some embodiments, the prostacyclin analog is iloprost. In some embodiments, the prostacyclin analog is cicaprost. In some embodiments, the prostacyclin analog is treprostinil.

In some embodiments, the allergic disease is selected from the group consisting of asthma, allergic rhinitis, allergic sinusitis, anaphylactic syndrome, urticaria, angioedema, atopic dermatitis, allergic contact dermatitis, erythema nodosum, and erythema multiforme. In some embodiments, the allergic disease is asthma.

In some embodiments, the prostacyclin or a prostacyclin analog is administered by a method selected from the group consisting of intravenous, oral, and aerosol.

In some embodiments, the administration of the prostacyclin or the prostacyclin analog decreases IL33 expression.

### Epoprostenol

Epoprostenol, a synthetic prostacyclin, is an unstable molecule having a half-life of about six minutes. Epoprostenol is commercially available from Actelion Pharmaceuticals (Veletri®) or from Glaxo-Smith Kline (Flolan®). Epoprostenol is diluted with sterile water to the desired concentration. Epoprostenol is the sodium salt of prostacyclin. Although the chemical is degraded by light and must be stored at a temperature below 25° C., it may be easily administered to a patient in need thereof.

Epoprostenol is administered intravenously. Due to the short half-life of the drug, and the fact that it is modified in the gastrointestinal tract, the drug is not a candidate to be ingested orally. The manufacturer recommends that the drug be reconstituted prior to use intravenously. Reconstitution for use is accomplished by adding a suitable carrier or diluent, which may be in the form of an aqueous or organic solution, dependent upon the manufacturer's instructions. Due to the instability of the drug, it should be used within a period of eight hours after it is reconstituted according to the instructions of the manufacturer.

Epoprostenol is provided to a patient intravenously. In certain embodiments, the infusion rate is initially from about 2 ng/kg/min to about 4 ng/kg/min. The infusion rate may be modified about every 15 minutes. The increase in rate of administration is limited by side effects (flushing, diarrhea, leg pain). The target dose is approximately from about 10 ng/kg/min to about 15 ng/kg/min and periodic dose increases are required to maximize efficacy. In some embodiments, the infusion rate is modified less frequently than every 15 minutes. In other embodiments, the infusion rate is modified more frequently than about every 15 minutes. Almost all clinical experience with epoprostenol has been limited to the treatment of pulmonary hypertension and in a survey of epoprostenol usage in the United States at nineteen different centers, the dose ranges were from 0.5-270 ng/kg/min. Recently, investigators experienced with the use of this agent suggested the optimal dose to be 22-45 ng/kg/min; lower doses seem to result in diminished effectiveness for pulmonary hypertension whereas higher doses result in excessive toxicity without increased effectiveness. Due to mobile intravenous pumps, a patient may receive epoprostenol intravenously for extended periods of time.

### Iloprost

Iloprost is an analog of prostacyclin/epoprostenol, but is more stable. Although the half-life is a relatively short (13 minutes), it is not degraded by light. Iloprost is commercially available from Actelion Pharmaceuticals (Ventavis®). Iloprost is diluted with sterile water to the desired concentration. Iloprost can be administered intravenously, orally, or by aerosol. Prior to administration, it is necessary to reconstitute the drug. Reconstitution for use is accomplished by adding a suitable carrier or diluent, which may be in the form of an aqueous or organic solution, dependent upon the manufacturer's instructions.

Subsequent to reconstitution, iloprost can be administered intravenously to a patient in need thereof. Administering iloprost for long periods of time does not appear harmful since studies involving the long-term administration of iloprost to pulmonary hypertension patients showed such administration to be safe.

In certain embodiments, iloprost may be provided to a patient intravenously. In such embodiments, the initial infusion rate is initially 2 ng/kg/min. In other embodiments, the initial infusion rate is from about 0.5 ng/kg/min to about 5 ng/kg/min. The infusion rate may be modified every 15 minutes. In certain embodiments, the infusion rate is modified less frequently than every 15 minutes. In still other embodiments, the infusion rate is modified more frequently than about every 15 minutes. In some embodiments, the increases to the infusion rate stop when the iloprost reaches 10 ng/kg/min. In certain embodiments, the increases to the infusion rate stop when the iloprost reaches from about 2 ng/kg/min to about 5 ng/kg/min. In other embodiments, the increases to the infusion rate stop when the iloprost reaches about 5 ng/kg/min. A patient may receive iloprost intravenously for extended periods of time.

Iloprost may also be administered orally. Although oral administration is possible, less than 20% of the iloprost will reach the systemic circulation. When administered orally, the iloprost dose can be 100 micrograms. In certain embodiments, the oral iloprost dose is from about 150 micrograms to about 50 micrograms. In other embodiments, the oral iloprost dose is about 50 micrograms. When iloprost is administered orally, a single dose can be given 2 times per day. In certain embodiments, the iloprost is administered at least two times per day. In other embodiments, the iloprost is administered no more than two times per day. A patient's reaction to the treatment is to be considered in order to modify the amount and/or frequency with which the patient receives iloprost.

In certain embodiments, iloprost is administered as an aerosol. In certain embodiments, an iloprost aerosol of about 50 micrograms/day is given. In other embodiments, an iloprost aerosol having a concentration of from about 200 micrograms/day to about 50 micrograms/day is given. When iloprost is given as an aerosol, doses should be given 6-12 times per day. In certain embodiments, the doses are given at a frequency of about 2 hours between doses. In other embodiments, while the aerosol treatment continues, at least one dose is given during each 4 hour period. In other embodiments, the aerosol is administered at least six times per 24 hour period. When iloprost is administered as an aerosol, it may be dispensed from any mobile or non-portable dispenser that is capable of discharging an aerosol. Examples of devices used to discharge aerosols include, but are not limited to, hand held dischargers, similar to those commonly used by asthma patients, emphysema patients, and patients requiring mechanical ventilation.

### Treprostinil

Treprostinil (also known as UT-15 or 15AU81), is another stable prostacyclin analog. Treprostinil has a longer half-life than epoprostenol. Treprostinil may be administered intravenously, subcutaneously, oral, or as an aerosol. Treprostinil has previously been safely used on patients to treat pulmonary hypertension. Patterson, et al., 1995, Am. J. Cardiol., 75:26A-33A. Treprostinil is commercially available from United Therapeutics, Silver Spring, Md. 20910 (Tyvaso®). Reconstitution of the drug is not necessary. Treprostinil is diluted into sterile water to the desired concentration.

Treprostinil may be administered intravenously to a patient in need thereof. In certain embodiments, the infusion rate is initially 10 ng/kg/min. In other embodiments, the initial infusion rate is from about 5 ng/kg/min to about 20 ng/kg/min. In still other embodiments, the initial infusion rate is 20 ng/kg/min. The infusion rate may be modified about every 15 minutes. In certain embodiments, the infusion rate is modified less frequently than every 15 minutes. In still other embodiments, the infusion rate is modified more frequently than every 15 minutes. In some embodiments, the increases to the infusion rate stop when the treprostinil reaches 10 ng/kg/min. In certain embodiments, the increases to the infusion rate stop when the treprostinil reaches from about 5 ng/kg/min to about 15 ng/kg/min. In other embodiments, the increases to the infusion rate stop when the treprostinil reaches 15 ng/kg/min.

Treprostinil may be administered orally to a patient in need thereof, and is available from United Therapeutics (Orenitram®). Treatment can be initiated with 0.125 mg TID (about 8 hours apart) or 0.25 mg BID (about 12 hours apart), and can then be titrated up or down every 3 to 4 days as needed.

Due to mobile devices used for distributing components intravenously, a patient may receive treprostinil intravenously for extended periods of time. Methods of drug delivery intravenously, for all components disclosed within this patent application, include microinfusion pumps.

Treprostinil may also be administered subcutaneously. Methods of administering treprostinil subcutaneously include, but are not limited to, subcutaneous inject with a needle and administration with a subcutaneous catheter. When administered subcutaneously, the treprostinil dose is about 10 ng/kg/min. In certain embodiments, the treprostinil dose is from about 5 ng/kg/min to about 20 ng/kg/min. In other embodiments, the treprostinil dose is 5 ng/kg/min. A patient's reaction to the treatment is to be considered in order to modify the amount and/or frequency with which the patient receives treprostinil.

Treprostinil can also be administered via inhalation. In one embodiment, administration is via inhalation via a metered dose inhaler (MDI) or nebulizer. In one embodiment, where compound delivery is via a nebulizer, the compound is provided to the patient as a composition, for example, as a lipid nanoparticle composition.

### Beraprost

Beraprost is a stable prostacyclin analog that may also be used in the methods disclosed herein. Beraprost is administered orally as beraprost sodium. Beraprost is commercially available from Astellas Pharma. The compound is available in tablets and administered orally. Oral ingestion for this compound, and other compounds, disclosed herein that are administered orally, is accomplished by ingesting either a solid structure (pill, or dissolvable coating containing the component) or a liquid solution containing the active component to be administered orally.

When administered orally, the beraprost dose can be about 60 micrograms per day. In certain embodiments, the oral beraprost dose is from about 60 micrograms/day to about 180 micrograms/day. In other embodiments, the beraprost dose is from about 90 micrograms/day to about 120 micrograms/day. In still other embodiments, the oral beraprost dose is from about 60 micrograms/day to about 90 micrograms/day.

When beraprost is administered orally, a single dose can be given about 4 times per day. In certain embodiments, the beraprost is administered at least 4 times per day. In other certain embodiments, the beraprost is administered more than 4 times per day. A patient's reaction to the treatment is to be considered in order to modify the amount and/or frequency with which the patient receives beraprost.

### Compositions

Compositions, as described herein, comprising an active compound and an excipient of some sort may be useful in a variety of applications.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semi-solid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfate, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, varoius gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacilic acid/acrylamide copolymer, polyalkylene oxides such as polyethylene oxide, polypropylene oxide, poly(ethylene oxide-propylene oxide), and a Pluronic polymer, polyoxyethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly(meth)acrylic acid, and esters amide and hydroxyalkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain embodiments, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The active ingredient may be administered in such amounts, time, and route deemed necessary in order to achieve the desired result. The exact amount of the active ingredient will vary from subject to subject, depending on the species, age, and general condition of the subject, the severity of the infection, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some embodiments, the active ingredient is administered via a variety of routes, including oral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

### PGI₂ Inhibits IL-33-Induced Th2 Differentiation, CD25 Expression and IL-2 Receptor Signaling in Mouse CD4⁺ T Cells

IL-33 has pleotropic functions in immune responses and promotes the development of inflammatory diseases. IL-33 induces Th2 differentiation and enhances type 2 cytokine production by CD4⁺ T cells. However, the regulation of IL-33-driven type 2 cytokine responses is not fully defined. In this example, the effect of PGI₂, a lipid mediator formed in the cyclooxygenase pathway of arachidonic acid metabolism, on naïve CD4⁺ T cell activation, proliferation and differentiation by IL-33 was investigated. Using wild type BALB/c mice and PGI₂ receptor (IP) knockout (KO) mice, it was found that the PGI₂ analog cicaprost dose-dependently inhibited IL-33-driven IL-4, IL-5, and IL-13 production by CD4⁺ T cells in an IP-specific manner. In addition, cicaprost suppressed the function of NFAT, a transcription factor that promotes IL-2 expression, and inhibited IL-33-driven IL-2 production by CD4⁺ T cells. Furthermore, cicaprost IP-dependently inhibited the expression of IL-2 receptor (IL-2R) α chain (CD25) and attenuated STAT5 phosphorylation. The suppressive effect of the PGI₂ analog cicaprost on IL-33-driven Th2 differentiation shows that PGI₂ limits the development of type 2 immune responses caused by IL-33-stimulating antigens such as protease-containing allergens.

### Background

The differentiation of naïve CD4⁺ T cells toward Th2 cells is required for defense against parasite pathogens, while dysfunctional type 2 immune responses may cause immunopathological diseases such as allergic diseases and asthma. Classical Th2 differentiation of CD4⁺ T cells is induced by IL-4 which activates the STAT6 signaling pathway for the expression of type 2 cytokines including IL-4, IL-5, and IL-13. IL-33 is a pro-inflammatory cytokine constitutively expressed in the nucleus of epithelial and endothelial cells and promotes innate immunity and adaptive type 2 immune responses (1-4). IL-33 binds to IL-1R1 (ST2) and increases IL-5 and IL-13 expression by mouse and human CD4⁺ T cells, indicating its ability to promote Th2 differentiation (5). The effect of IL-33 on naïve CD4⁺ T cell Th2 differentiation is mediated by the MyD88 signaling pathway, but is independent of IL-4, IL-4R, and STAT6 signaling (5). Therefore, IL-33 activates an alternative Th2 differentiation pathway from the classical IL-4-driven STAT6-dependent Th2 cell polarization.

Lipid molecules such as prostaglandins formed in the cyclooxygenase pathway of arachidonic acid metabolism have regulatory functions in immune responses and inflammation (6-9). PGI₂, also known as prostacyclin, suppressed Th2 cytokine (IL-5 and IL-13) expression, eosinophilia, and mucus production in the lung through PGI₂ receptor (IP) signaling pathway in a mouse model of OVA-induced allergic inflammation (10-13). Recently, it was reported that IP deficiency abrogated OVA-induced immune tolerance in mice, further supporting the modulatory functions of PGI₂ not only on immune responses, but also on immune tolerance (14). In vitro, the PGI₂ analogs cicaprost and iloprost inhibited the production of the effector cytokines IL-4 and IL-5 by Th2 cells that had been differentiated under IL-4 and anti-IFN-γ condition (15). However, the effect of PGI₂ on IL-33-driven Th2 polarization has not been reported.

In this example, PGI₂ inhibition IL-33-induced CD4⁺ T cell activation, proliferation and type 2 cytokine expression was investigated. It was found that the PGI₂ analog cicaprost suppressed the production of IL-4, IL-5 and IL-13 by WT CD4⁺ T cells, but not by IP KO CD4⁺ T cells. In addition, cicaprost signaling through IP decreased IL-2 production, CD25 surface expression, and IL-2 signaling by suppressing STAT5 phosphorylation.

### Materials and Methods

### Mice

Wild type (WT) BALB/c mice were obtained from The Jackson Laboratory (Bar Harbor, Me.). IP KO mice on a C57BL/6 background were generated by homologous recombination in embryonic stem cells and kindly provided by Dr. Garret FitzGerald at the University of Pennsylvania. The IP KO mice were backcrossed to a BALB/c background for 10 generations. Age-matched WT and IP KO mice were used at 8-12 weeks old. Animal experiments were reviewed, approved by the Institutional Animal Care and Use Committee at Vanderbilt University, and were conducted according to the guidelines for the Care and Use of Laboratory Animals prepared by the Institute of Laboratory Animal Resources, National Research Council.

### CD4⁺ T Cell Culture

CD4⁺ T cells were purified from the splenic cells of WT, or IP KO mice by a mouse naive CD4⁺ T cell isolation kit II (Miltenyi Biotec, Auburn, Calif.). The purified CD4⁺CD62L⁺ T cell population contains 97.5% CD3⁺CD4⁺ cells as determined by flow cytometry. The purified CD4 T cells were resuspended at 1×10⁶ cells/ml in RPMI 1640 medium (Mediatech, Herndon, Va.) supplemented with 10% FBS (HyClone, Logan, Utah), 4 mM of L-glutamine, 1 mM of sodium pyruvate, 55 mM of 2-ME, 10 mM of HEPES, 100 U/ml penicillin, and 100 μg/ml streptomycin. The cells were stimulated with plate-bound anti-CD3 (10 μg/ml) and anti-CD28 (2 μg/ml; BD Biosciences, San Diego, Calif.) in 96-well flat-bottom plates and treated with IL-33 (20 ng/ml) plus cicaprost (a generous gift provided by Dr. Manuela Huebner, Bayer HealthCare, Berlin, Germany) at various concentrations or vehicle (water) control. The cells were cultured for 3 days. For T cell proliferation experiments, naive CD4⁺ T cells were stained with 1 μM CFSE before cell culture and cicaprost treatment.

Cell culture supernatant was collected at day 2 for IL-2 measurement because IL-2 production by activated CD4⁺ T cells peak at day 2 (unpublished data). At day 3, the culture supernatant was harvested for IL-4, IL-5 and IL-13 measurement. At day 2, the cells were harvested and the cell lysate was prepared for p-STAT5 measurement.

### NFAT Function Assay

To determine NFAT activation, a Cignal Lenti NFAT reporter (luc) kit (Qiagen, Hilden, Germany), was used to transduce naïve CD4⁺ T cells. CD4⁺ T cells (10⁵) of WT and IP KO mice were transduced with 2×10⁵ transduction units of lentiviral vector (MOI=2) and 30 μg transduction reagent in 60 μl of transduction medium without antibiotics for 24 h in 96 well plates coated with anti-CD3 and anti-CD28. The cells were then pooled, washed, seeded and treated with IL-33 plus either cicaprost or vehicle. After culture for 2 more days in the same plates, the cells were harvested and the cell lysate was used for luciferase assay with Luciferase Assay System with Reporter Lysis Buffer (Promega, Madison, Wis.). The luciferase activity indicates the levels of NFAT activation.

### Flow Cytometry

At day 3 of cell culture, the cells were harvested for flow cytometric staining with fluorochrome-labeled antibodies against CD4⁺ (Biolegend, San Diego, Calif.), Gata3 (Affymetrix, Santa Clara, Calif.), CD25 (BD Biosciences, San Jose, Calif.), or TSLPR (R&D Systems, Minneapolis, Minn.). An LSR II flow cytometer (BD Biosciences) was used for flow cytometry and the data were analyzed with FlowJo software (FlowJo, LLC, Ashland Oreg.). For cell proliferation assay, CFSE intensity of the cultured cells was determined at day 3 by flow cytometry. Cell division index was calculated with FlowJo software. At day 3, the cells were stained with the cell viability dye 4,6-diamidino-2-phenylindole (DAPI), and 123count™ eBeads (Affymetrix) were added to the cell solution for live cell counting by flow cytometry.

### ELISA

IL-2, IL-4, IL-5, and IL-13 in the culture supernatant were measured by DuoSet ELISA kits from R&D Systems according to the manufacturer's instructions. Phosphorylated STAT5 (p-STAT5) was measured by phosphor-STAT5A/B (Tyr694/Tyr699) InstantOne™ ELISA kit (Affymetrixes). Measurements below the limit of detection were assigned a value of half the lower limit of detection for purposes of statistical analyses.

### Statistical Analysis

The results were presented as mean±SEM. Statistical analyses were conducted by using one-way ANOVA with a Bonferroni post hoc test.

### Results

### The PGI₂ Analog Cicaprost Decreased IL-33-Induced Type 2 Cytokine Production by CD4⁺ T Cells

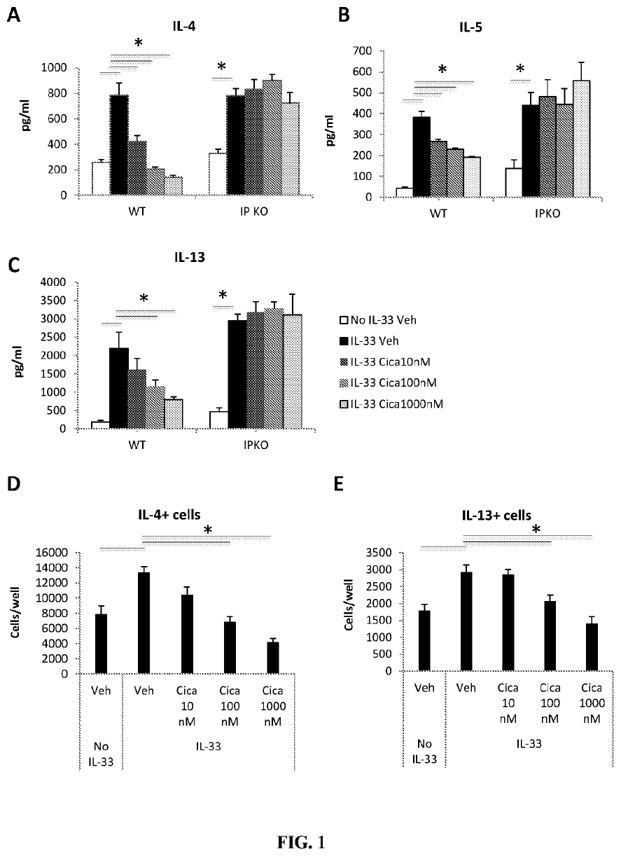

To determine the effect of PGI₂ signaling on IL-33-induced Th2 differentiation, naïve CD4⁺ T cells of WT and IP KO mice, both on BALB/c background, were activated with anti-CD3 and anti-CD28 either with or without IL-33 and treated with cicaprost or vehicle for 3 days. Compared to the cell culture without IL-33, IL-33 significantly increased the production of the type 2 cytokines IL-4, IL-5, and IL-13 (FIG. 1, A-C). In the presence of IL-33, cicaprost dose dependently decreased IL-4, IL-5 and IL-13 production by WT CD4⁺ T cells, showing that PGI₂ has an inhibitory effect on Th2 differentiation and type 2 cytokine production. Cicaprost did not change the production of IL-4, IL-5, or IL-13 by IP KO CD4⁺ T cells, indicating that cicaprost-mediated inhibition of type 2 cytokine production is dependent on IP signaling.

To determine the effect of cicaprost on the total number of cells that produce type 2 cytokines, the purified T cells were cultured and treated with vehicle or cicaprost for 3 days and performed intracellular cytokine staining for flow cytometry. It was found that IL-33 increased the numbers of IL-4⁺ T cells and IL-13⁺ T cells compared with the cell culture in the absence of IL-33 (FIGS. 1D and 1E). In the presence of IL-33, cicaprost dose-dependently decreased total numbers of IL-4⁺ T cells and IL-13⁺ T cells compared with vehicle control (FIGS. 1D and 1E).

### Cicaprost Decreased Gata3 Expression in CD4⁺ T Cells

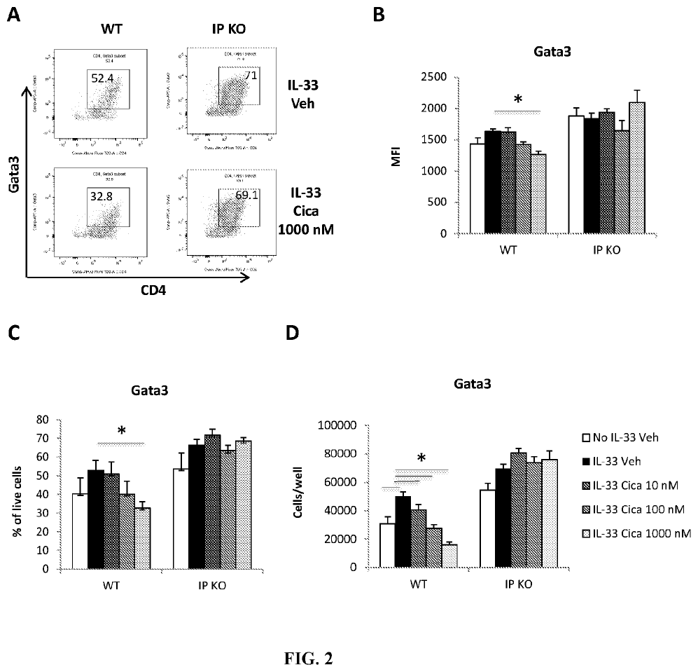

The differentiation of naïve CD4⁺ T cells into Th2 cells under IL-4 culture conditions requires the expression of the transcription factor Gata3 (18). To investigate whether Gata3 plays a role in the inhibitory effect of cicaprost on IL-33-induced type 2 cytokine production, the expression of Gata3 in CD4⁺ T cells activated with IL-33 and treated with cicaprost at various concentrations or vehicle for 3 days was determined. It was found that cicaprost at 1000 nM significantly decreased Gata3 mean fluorescence intensity (FIG. 2B), the percentages (FIG. 2C), and total number (FIG. 2D) of Gata3-expressing CD4⁺ T cells from WT mice, but not IP KO mice (FIG. 2). Therefore, the IP-dependent suppression of type 2 cytokine production by cicaprost was associated with decreased Gata3 expression, showing that PGI₂/IP signaling inhibits Gata3 expression and suppresses Th2 differentiation.

### Cicaprost Decreased IL-2 Production, NFAT Activation, and CD4⁺ T Cell Activation and Proliferation

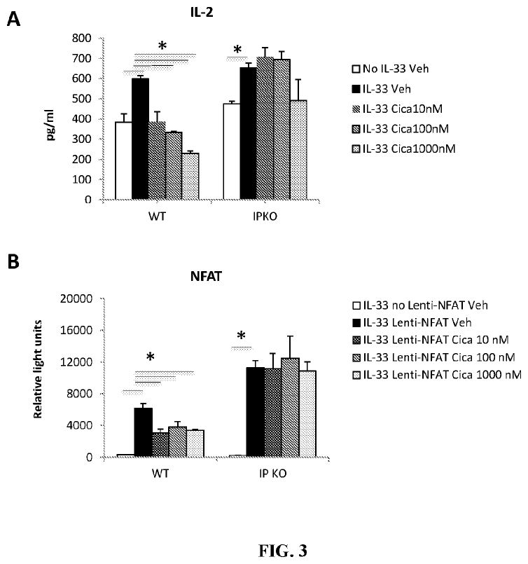

In vitro activation of mouse naïve CD4⁺ T cells results in the expression of IL-2, a growth factor critical for cell survival and proliferation (19). To investigate whether cicaprost inhibits IL-33-induced type 2 cytokine production by suppressing CD4⁺ T cell IL-2 production and cell activation, IL-2 levels were measured in the culture supernatant. It was found that IL-33 significantly increased CD4⁺ T cell IL-2 expression (FIG. 3A). Cicaprost dose-dependently suppressed IL-2 production by WT CD4⁺ T cells, but not by IP KO CD4⁺ T cells, indicating that the suppressive effect of cicaprost is dependent on IP signaling (FIG. 3A).

To determine whether the suppressive effect of cicaprost on IL-2 production is associated with the inhibition of NFAT, a transcription factor that binds to the IL-2 gene promoter to activate IL-2 gene expression, an NFAT function assay was performed with an NFAT-luciferase reporter lentiviral vector system. It was found that cicaprost significantly suppressed NFAT function in WT cells, but not IP KO cells after 3 days of the cell culture (FIG. 3B). These results show that cicaprost could inhibit IL-2 production by attenuating NFAT activation.

To further analyze the effect of cicaprost on cell proliferation, naïve CD4⁺ T cells were CFSE-labeled, activated with anti-CD3 and anti-CD28, and treated with IL-33 plus either vehicle or cicaprost. Cell proliferation was determined by CFSE dilution. As shown in FIG. 4, cicaprost at 1000 nM significantly decreased the cell division index and total number of live cells of WT CD4⁺ T cells, but not of IP KO CD4⁺ T cells (FIG. 4). These data indicate that cicaprost inhibited cell activation and proliferation in an IP-dependent manner.

### Cicaprost Decreased IL-5 and IL-13 Production by CD4⁺ T Cells in the Presence of Exogenous IL-2

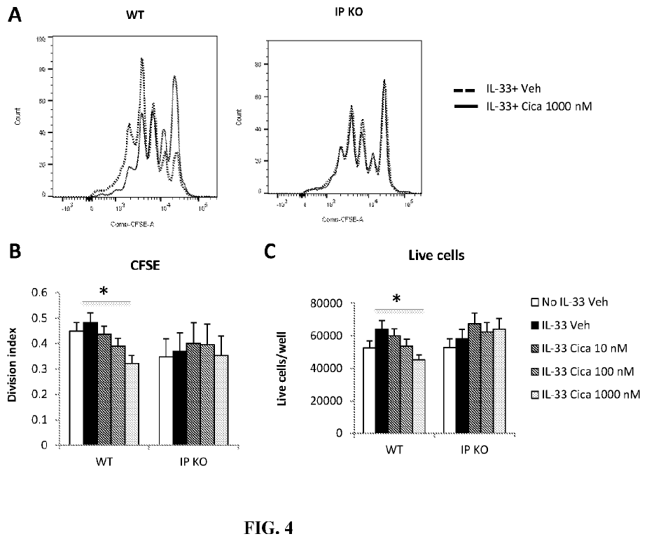

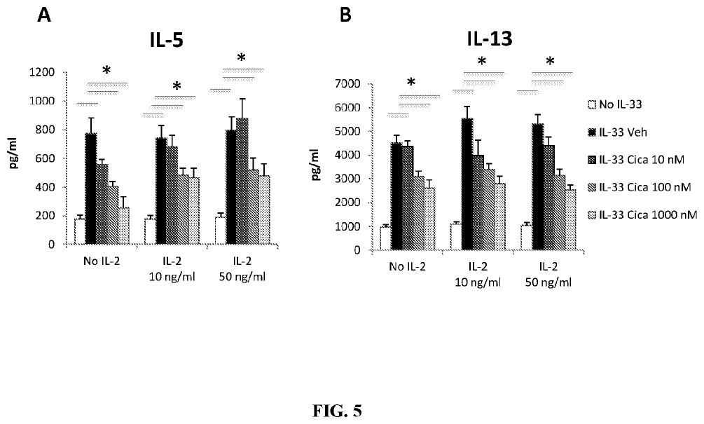

IL-2 not only stimulates CD4⁺ T cell survival and expansion, but also promotes Th2 differentiation in the presence of IL-4 and anti-IFN-γ through the STAT5 signaling pathway (20). The correlation between IL-2 inhibition and type 2 cytokine suppression in cicaprost-treated cells shows that decreased IL-2 expression may be a mechanism by which cicaprost subsequently inhibits IL-33-induced type 2 cytokine production. To test this, IL-2 was added to the cell cultures, reasoning that if cicaprost inhibits type 2 cytokine production by affecting IL-2 production, addition of exogenous IL-2 should attenuate or abrogate the effect of cicaprost. It was found that cicaprost still significantly inhibited IL-5 and IL-13 production in the presence of either 10 ng/ml or 50 ng/ml IL-2 (FIGS. 5A and 5B), showing that other mechanisms and molecular pathways are involved in the inhibitory effect of cicaprost on type 2 cytokine production.

### Cicaprost Decreased CD25 Expression on CD4⁺ T Cells

To determine whether cicaprost attenuates the IL-2 signaling pathway, CD4⁺ T cells activated and treated with either cicaprost or vehicle for 3 days were stained with fluorochrome-labeled anti-CD25 antibody and analyzed by flow cytometry. It was found that cicaprost dose-dependently decreased CD25 mean fluorescence intensity, and the percentages and total numbers of CD25 expressing cells compared with vehicle control (FIG. 6 A-D). As a comparison, cicaprost did not change TSLPR MFI and percentages of TSLPR expressing cells in WT or IP KO T cell culture in the presence of IL-33 (data not shown).

Binding of IL-2 to IL-2 receptor leads to the activation of JAK1 and JAK3 and the activation of the downstream transcription factors STAT5 (21). To test whether cicaprost downregulates IL-2 receptor signaling, the levels of phosphorylated STAT5 were measured in CD4⁺ T cells harvested 2 days after the cell activation and treatment with IL-33 plus either vehicle or cicaprost. Cicaprost dose-dependently attenuated STAT5 phosphorylation in WT cells, but not in IP KO cells, indicating an IP-specific inhibition of IL-2 receptor signaling by cicaprost (FIG. 6E).

### Discussion

The discovery of IL-33-induced Th2 polarization reveals an alternative Th2 differentiation pathway other than the classical IL-4-driven Th2 differentiation pathway (5). Earlier findings were published on the regulation of allergic responses and inflammation by lipid mediators formed in arachidonic acid metabolism (10, 14, 22). It has been shown that PGI₂ and IP signaling inhibit the effector cytokines production by Th2 cells that had been differentiated under classical Th2 conditions with IL-4 and anti-IFN-γ (15). In this example, it was demonstrated that the PGI₂ analog cicaprost and IP signaling negatively regulated IL-33-driven Th2 differentiation and reduced IL-4, IL-5, and IL-13 production and Gata3 expression in CD4⁺ T cells in a dose-dependent manner. The inhibitory effect of cicaprost is associated with decreased NFAT activation, IL-2 expression, CD4⁺ T cell activation, and cell division, and correlated with lower surface expression of IL-2Rα chain (CD25) and STAT5 phosphorylation in CD4⁺ T cells. Therefore, in addition to the inhibition of IL-4-driven classical Th2 differentiation, the data presented here shows that IP signaling restrains IL-33-driven alternative Th2 differentiation in part by inhibiting IL-2 production and suppressing IL-2R expression and signaling activities.

Naïve CD4⁺ T cells activated with anti-CD3 and stimulated by IL-33 produced IL-5 and IL-13, but not IL-4 (5). In this example, it was found that IL-33 stimulated not only IL-5 and IL-13 expression, but also IL-4 production by either naïve WT BALB/c or IP KO CD4⁺ T cells after activation with anti-CD3 and anti-CD28. The difference in the findings may be explained by variations in the presence of costimulatory signals and TCR stimulation strength. In addition to studies focusing on the stimulatory functions of IL-33 on innate and adaptive type 2 responses (5, 23), more recent reports showed that IL-33 can act as an enhancer for effector cytokine production by Th1 cells, Th17, and Treg cells (24-26). The data here demonstrate that PGI₂ and IP signaling restrain IL-33-induced Th2 differentiation, expansion, and type 2 cytokine production.

It has been reported that the augmentation of IL-5 and IL-13 production by IL-33 is not dependent on IL-4 and STAT6, as IL-33 enhanced IL-5 and IL-13 expression in IL-4 KO and STAT6 KO CD4⁺ T cells and IL-33-induced Th2 differentiation was not mediated by the IL-4R signaling molecule Gata3 (5). In this example, IL-33 did not increase Gata3 expression in either WT or IP KO CD4⁺ T cells, supporting a marginal role of Gata3 in IL-33-induced Th2 differentiation. It was found that cicaprost-mediated suppression of Th2 differentiation is associated with the down-regulation of Gata3 protein expression, suggesting that the baseline Gata3 expression is important for type 2 cytokine production in the presence of IL-33.

This data shows that cicaprost suppressed IL-33-driven Th2 differentiation partially by inhibiting IL-2R CD25 expression and attenuating IL-2R signaling. IL-2 is a critical cytokine for CD4⁺ T cell activation and clonal expansion through autocrine and paracrine signaling. IL-33 signaling promoted STAT5 mRNA expression and phosphorylation in response to IL-2 (24). In agreement with these findings, it was demonstrated that IL-33 significantly enhanced IL-2 production in WT and IP KO CD4⁺ T cells and cicaprost dose-dependently inhibited IL-33-induced IL-2 production in WT CD4⁺ T cells, but not in IP KO CD4⁺ T cells. Furthermore, cicaprost suppressed the IL-2R α chain CD25 expression. Since IL-2 contributes to CD4⁺ T cell expression and clonal expansion, the down-regulation of both IL-2 and IL-2R may contribute to cicaprost-mediated suppression of IL-2R signaling and Th2 differentiation. Indeed, inhibition of STAT5 phosphorylation, a transcription factor in IL-2 signaling pathway, was observed in cicaprost-treated WT CD4⁺ T cells, but not in IP KO CD4⁺ T cells.

By using WT and IP KO mice, it was demonstrated that cicaprost-mediated inhibition of IL-33-induced Th2 differentiation, type 2 cytokine production, IL-2 production, CD25 expression, and STAT5 activation are IP-dependent. These data show that PGI₂ signaling through IP restrains IL-33-induced Th2 differentiation and type 2 cytokine responses in adaptive immune cells. Recent reports have shown that cicaprost inhibited IL-33-induced cytokine responses of type 2 innate lymphoid cells (ILC2) (27). Using a mouse model of Alternaria alternata extract-induced and IL-33-dependent lung inflammation, it was revealed that endogenous PGI₂ has inhibitory function on ILC2 cells (27). It was demonstrated that IP-deficiency resulted in greater ILC2 responses compared to ILC2 responses in WT mice, and conversely cicaprost inhibited ILC2 response in WT mice (27). Based on the results here, it appears that PGI₂ and IP receptor signaling limits IL-33- and IL-4-induced Th2 cell responses, as well as ILC2 function, highlighting the broad regulatory properties of PGI₂ in adaptive and innate immunity and inflammation.

In summary, the data presented here indicate that PGI₂ analogs significantly down-regulate IL-33-induced Th2 differentiation and type 2 cytokine production, via suppressing IL-2 production, CD25 expression, IL-2R signaling, and Gata3 expression. These results provide the first evidence showing that PGI₂/IP signaling suppresses IL-33-induced Th2 differentiation. This example broadens the understanding of PGI₂ on immune responses, considering previous findings showing the inhibitory effect of PGI₂ on IL-4-induced Th2 effector cytokine production and on IL-33-induced ILC2 responses. IL-33 plays an important role in the development of allergic diseases induced by protease-containing allergens such as Alternaria alternata (2, 28). PGI₂ analog-mediated inhibition of IL-33-induced Th2 differentiation shows that PGI₂ limits Th2 differentiation and allergy development induced by protease-containing allergens. PGI₂ and its analogs (for example, iloprost and treprostinil) are Food and Drug Administration (FDA)-approved drugs for the clinical use to treat pulmonary hypertension (29), and can also be used for treating and/or preventing IL-33-related type 2 immune disorders such as allergic diseases and asthma.

### REFERENCES CITED IN THIS EXAMPLE

1. Moussion, C., N. Ortega, and J. P. Girard. 2008. The IL-1-like cytokine IL-33 is constitutively expressed in the nucleus of endothelial cells and epithelial cells in vivo: a novel ‘alarmin’? PLoS One 3: e3331.
2. Iijima, K., T. Kobayashi, K. Hara, G. M. Kephart, S. F. Ziegler, A. N. McKenzie, and H. Kita. 2014. IL-33 and thymic stromal lymphopoietin mediate immune pathology in response to chronic airborne allergen exposure. J Immunol 193: 1549-1559.
3. Carriere, V., L. Roussel, N. Ortega, D. A. Lacorre, L. Americh, L. Aguilar, G. Bouche, and J. P. Girard. 2007. IL-33, the IL-1-like cytokine ligand for ST2 receptor, is a chromatin-associated nuclear factor in vivo. Proc Natl Acad Sci USA 104: 282-287.
4. Halim, T. Y., C. A. Steer, L. Matha, M. J. Gold, I. Martinez-Gonzalez, K. M. McNagny, A. N. McKenzie, and F. Takei. 2014. Group 2 innate lymphoid cells are critical for the initiation of adaptive T helper 2 cell-mediated allergic lung inflammation. Immunity 40: 425-435.
5. Kurowska-Stolarska, M., P. Kewin, G. Murphy, R. C. Russo, B. Stolarski, C. C. Garcia, M. Komai-Koma, N. Pitman, Y. Li, W. Niedbala, A. N. McKenzie, M. M. Teixeira, F. Y. Liew, and D. Xu. 2008. IL-33 induces antigen-specific IL-5+ T cells and promotes allergic-induced airway inflammation independent of IL-4. J Immunol 181: 4780-4790.
6. Peebles, R. S., Jr., K. Hashimoto, J. D. Morrow, R. Dworski, R. D. Collins, Y. Hashimoto, J. W. Christman, K. H. Kang, K. Jarzecka, J. Furlong, D. B. Mitchell, M. Talati, B. S. Graham, and J. R. Sheller. 2002. Selective cyclooxygenase-1 and -2 inhibitors each increase allergic inflammation and airway hyperresponsiveness in mice. Am. J. Respir. Crit Care Med. 165: 1154-1160.
7. Cheng, J., R. T. Dackor, J. A. Bradbury, H. Li, L. M. DeGraff, L. K. Hong, D. King, F. B. Lih, A. Gruzdev, M. L. Edin, G. S. Travlos, G. P. Flake, K. B. Tomer, and D. C. Zeldin. 2016. Contribution of alveolar type II cell-derived cyclooxygenase-2 to basal airway function, lung inflammation, and lung fibrosis. FASEB journal: official publication of the Federation of American Societies for Experimental Biology 30: 160-173.
8. Jaffar, Z., M. E. Ferrini, M. C. Buford, G. A. FitzGerald, and K. Roberts. 2007. Prostaglandin I2-IP signaling blocks allergic pulmonary inflammation by preventing recruitment of CD4+ Th2 cells into the airways in a mouse model of asthma. J. Immunol. 179: 6193-6203.
9. Idzko, M., H. Hammad, M. van Nimwegen, M. Kool, N. Vos, H. C. Hoogsteden, and B. N. Lambrecht. 2007. Inhaled iloprost suppresses the cardinal features of asthma via inhibition of airway dendritic cell function. The Journal of clinical investigation 117: 464-472.
10. Zhou, W., J. Zhang, K. Goleniewska, D. E. Dulek, S. Toki, D. C. Newcomb, J. Y. Cephus, R. D. Collins, P. Wu, M. R. Boothby, and R. S. Peebles, Jr. 2016. Prostaglandin I2 Suppresses Proinflammatory Chemokine Expression, CD4 T Cell Activation, and STAT6-Independent Allergic Lung Inflammation. J Immunol 197: 1577-1586.
11. Nagao, K., H. Tanaka, M. Komai, T. Masuda, S. Narumiya, and H. Nagai. 2003. Role of prostaglandin I2 in airway remodeling induced by repeated allergen challenge in mice. Am. J. Respir. Cell Mol. Biol. 29: 314-320.
12. Takahashi, Y., S. Tokuoka, T. Masuda, Y. Hirano, M. Nagao, H. Tanaka, N. Inagaki, S. Narumiya, and H. Nagai. 2002. Augmentation of allergic inflammation in prostanoid IP receptor deficient mice. Br. J. Pharmacol. 137: 315-322.
13. Jaffar, Z., K. S. Wan, and K. Roberts. 2002. A key role for prostaglandin I2 in limiting lung mucosal Th2, but not Th1, responses to inhaled allergen. J. Immunol. 169: 5997-6004.
14. Zhou, W., K. Goleniewska, J. Zhang, D. E. Dulek, S. Toki, M. T. Lotz, D. C. Newcomb, M. G. Boswell, V. V. Polosukhin, G. L. Milne, P. Wu, M. L. Moore, G. A. FitzGerald, and R. S. Peebles. 2014. Cyclooxygenase inhibition abrogates aeroallergen-induced immune tolerance by suppressing prostaglandin I2 receptor signaling. The Journal of allergy and clinical immunology 134: 698-70500000.
15. Zhou, W., T. S. Blackwell, K. Goleniewska, J. F. O'Neal, G. A. FitzGerald, M. Lucitt, R. M. Breyer, and R. S. Peebles, Jr. 2007. Prostaglandin I2 analogs inhibit Th1 and Th2 effector cytokine production by CD4 T cells. J. Leukoc. Biol. 81: 809-817.
16. Cheng, Y., S. C. Austin, B. Rocca, B. H. Koller, T. M. Coffman, T. Grosser, J. A. Lawson, and G. A. FitzGerald. 2002. Role of prostacyclin in the cardiovascular response to thromboxane A2. Science 296: 539-541.
17. Price, A. E., H. E. Liang, B. M. Sullivan, R. L. Reinhardt, C. J. Eisley, D. J. Erle, and R. M. Locksley. 2010. Systemically dispersed innate IL-13-expressing cells in type 2 immunity. Proc Natl Acad Sci USA 107: 11489-11494.
18. Pai, S. Y., M. L. Truitt, and I. C. Ho. 2004. GATA-3 deficiency abrogates the development and maintenance of T helper type 2 cells. Proc. Natl. Acad. Sci. U.S.A 101: 1993-1998.
19. Sojka, D. K., D. Bruniquel, R. H. Schwartz, and N. J. Singh. 2004. IL-2 secretion by CD4+ T cells in vivo is rapid, transient, and influenced by TCR-specific competition. J Immunol 172: 6136-6143.
20. Cote-Sierra, J., G. Foucras, L. Guo, L. Chiodetti, H. A. Young, J. Hu-Li, J. Zhu, and W. E. Paul. 2004. Interleukin 2 plays a central role in Th2 differentiation. Proc Natl Acad Sci USA 101: 3880-3885.
21. Malek, T. R. 2008. The biology of interleukin-2. Annu Rev Immunol 26: 453-479.
22. Zhou, W., D. C. Newcomb, M. L. Moore, K. Goleniewska, J. F. O'Neal, and R. S. Peebles, Jr. 2008. Cyclooxygenase inhibition during allergic sensitization increases STAT6-independent primary and memory Th2 responses. J. Immunol. 181: 5360-5367.
23. Schmitz, J., A. Owyang, E. Oldham, Y. Song, E. Murphy, T. K. McClanahan, G. Zurawski, M. Moshrefi, J. Qin, X. Li, D. M. Gorman, J. F. Bazan, and R. A. Kastelein. 2005. IL-33, an interleukin-1-like cytokine that signals via the IL-1 receptor-related protein ST2 and induces T helper type 2-associated cytokines. Immunity 23: 479-490.
24. Guo, L., G. Wei, J. Zhu, W. Liao, W. J. Leonard, K. Zhao, and W. Paul. 2009. IL-1 family members and STAT activators induce cytokine production by Th2, Th17, and Th1 cells. Proc Natl Acad Sci USA 106: 13463-13468.
25. Baumann, C., W. V. Bonilla, A. Frohlich, C. Helmstetter, M. Peine, A. N. Hegazy, D. D. Pinschewer, and M. Lohning. 2015. T-bet- and STAT4-dependent IL-33 receptor expression directly promotes antiviral Th1 cell responses. Proc Natl Acad Sci USA 112: 4056-4061.
26. Schiering, C., T. Krausgruber, A. Chomka, A. Frohlich, K. Adelmann, E. A. Wohlfert, J. Pott, T. Griseri, J. Bollrath, A. N. Hegazy, O. J. Harrison, B. M. Owens, M. Lohning, Y. Belkaid, P. G. Fallon, and F. Powrie. 2014. The alarmin IL-33 promotes regulatory T-cell function in the intestine. Nature 513: 564-568.
27. Zhou, W., S. Toki, J. Zhang, K. Goleniewksa, D. C. Newcomb, J. Y. Cephus, D. E. Dulek, M. H. Bloodworth, M. T. Stier, V. Polosuhkin, R. D. Gangula, S. A. Mallal, D. H. Broide, and R. S. Peebles, Jr. 2016. Prostaglandin I2 Signaling and Inhibition of Group 2 Innate Lymphoid Cell Responses. American journal of respiratory and critical care medicine 193: 31-42.
28. Kita, H. 2015. ILC2s and fungal allergy. Allergol Int 64: 219-226.
29. Dorris, S. L., and R. S. Peebles. 2011. PGI2 as a regulator of inflammatory diseases. Mediators of inflammation 2012: 926968.

### Example 2

### Prostacyclin Analogs for Preventing Organ Graft Rejection

### Background

Solid organ transplantation is a life-changing therapy for end stage diseases of the heart, kidney, lung, liver, and pancreas. Major barriers to long-term transplant graft survival are acute and chronic rejection, processes which are largely mediated by T and B lymphocytes that cause graft dysfunction. Immunosuppressive therapy is a major strategy to decrease T cell-mediated rejection; however, there are significant side effects that complicate their use. Calcineurin inhibitors are a widely used component of current immunosuppressive regimens to prevent T lymphocyte-mediated rejection. Calcineurin inhibitors, such as cyclosporine and tacrolimus prevent activation of nuclear factor of activated T cells, cytoplasmic 1 (NFATc) and thus inhibit transcription of the IL2 gene, resulting in the beneficial immunosuppressive effects of this class of medications to prevent transplant rejection. However, calcineurin inhibitors have significant adverse effects such as hypertension and renal dysfunction that may limit their use, thus increasing the risk of graft rejection. Thus, a pharmacologic agent that inhibits NFATc activation, yet that does not have the deleterious side effects that occur with the use of calcineurin inhibitors, would be a major therapeutic advance in preventing solid organ graft rejection.

In this example, the data below reveals that prostaglandin (PG) I₂, also known as prostacyclin, reduces NFATc activation and IL-2 production that is specific to signaling through its G protein-coupled receptor, known as IP. Additional data reveals that PGI₂ signaling through IP enhances T regulatory cell (Treg) function, and Treg are critical to prevent rejection in mouse models of allograft transplantation. Further, the interaction between MHC class II and CD4+ T cells is critical for the immune destruction of grafted organs. In published studies in allergen challenge models, it was reported that PGI₂ signaling through IP significantly blunted antigen-specific dendritic cell activation of CD4+ cells, reduced dendritic cell MHC II and costimulatory molecule expression, directly inhibited T cell effector function, and promoted immune tolerance in mice.

As discussed below, PGI₂ signaling through IP can ameliorate solid organ graft rejection. This represents a paradigm shift in understanding how to prevent solid organ transplant rejection, as PGI₂ has a more favorable side effect profile than calcineurin inhibitors, thus making it a superior alternative to calcineurin inhibition therapy. This result has important therapeutic implications as PGI₂ and PGI₂ analogs are currently approved by the Food and Drug Administration (FDA) for the treatment of pulmonary hypertension and therefore could be re-purposed to prevent solid organ graft rejection.

PGI₂ is a product of the cyclooxygenase pathway of arachidonic acid metabolism. PGI₂ was discovered in 1976 as a result of its protective effects on endothelial cell function, relaxation of isolated blood vessels, and inhibition of platelet aggregation that limits thrombosis, and is approved by the Food and Drug Administration (FDA) for the treatment of pulmonary hypertension. For many years, PGI₂ was administered to patients by continuous intravenous infusion because of its heat lability that results in a short biologic half-life of only 46 seconds. Complications from continuous intravenous infusions led to the development of PGI₂ analogs that were administered by inhalation, resulting in the FDA approval of iloprost (2004) and treprostinil (2009); however, these inhaled agents had to be administered every 4 hours, thus being inconvenient for patient care and reducing patient compliance. The FDA approved an oral PGI₂ analog, treprostinil, for the treatment of pulmonary hypertension in 2013. Oral treprostinil dosing is twice per day, thus making PGI₂-based therapy more convenient. Thus, PGI₂ has been used clinically for over 30 years, has a strong safety profile.

The beneficial effects of PGI₂ on vascular function, including thrombosis prevention, along with its immunosuppressive effects on T and B lymphocytes, can ameliorate both acute and chronic solid organ graft rejection. Acute rejection results from vascular and parenchymal injury mediated by T cells and antibodies, and this process may start as early as one week after transplantation. T cells have a primary role in acute rejection by reacting to alloantigens, particularly MHC molecules that are present on vascular endothelial and parenchymal cells. These activated T cells can directly kill graft cells, or may produce effector cytokines that recruit and activate other inflammatory cells that injure the graft. In highly vascular grafts such as heart and kidneys, endothelial cells are the earliest targets of acute rejection. Chronic rejection is a result of graft vasculopathy that occurs with arterial occlusion from proliferation of intimal smooth muscle cells, leading to graft failure due to ischemic changes. With progression of graft arteriosclerosis, blood flow to the graft parenchyma is compromised and the parenchyma is slowly replaced by nonfunctioning fibrous tissue. Several growth factors and cytokines are implicated in graft fibrosis, including fibroblast growth factor, TGF-β, and IL-13. Alloantibodies also have an important role in acute rejection as they bind alloantigens, predominantly HLA molecules, on the vascular endothelial cells, leading to endothelial injury and intravascular thrombosis that result in graft injury. With advances in the treatment of acute rejection, graft vasculopathy and chronic rejection have become a major cause of allograft loss.

PGI₂ has been shown to be a critical mediator of immune tolerance. In data shown below, it was found that PGI₂ critically regulates T regulatory cell (Treg) function and modulates Foxp3, a member of the forkhead family of transcription factors that is critical for the development and function of most Tregs.

### PGI₂ Signaling Effects on Treg Foxp3 Expression and Suppressive Function

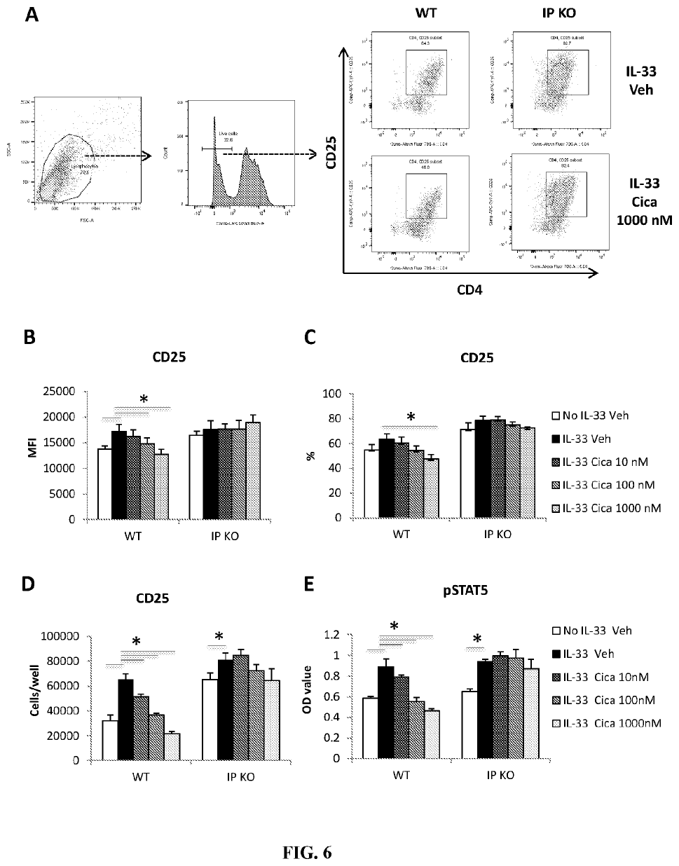

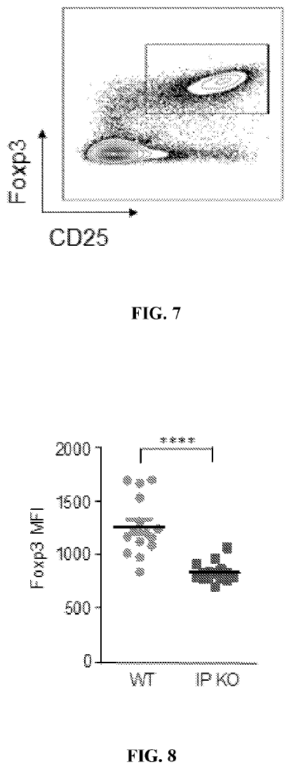

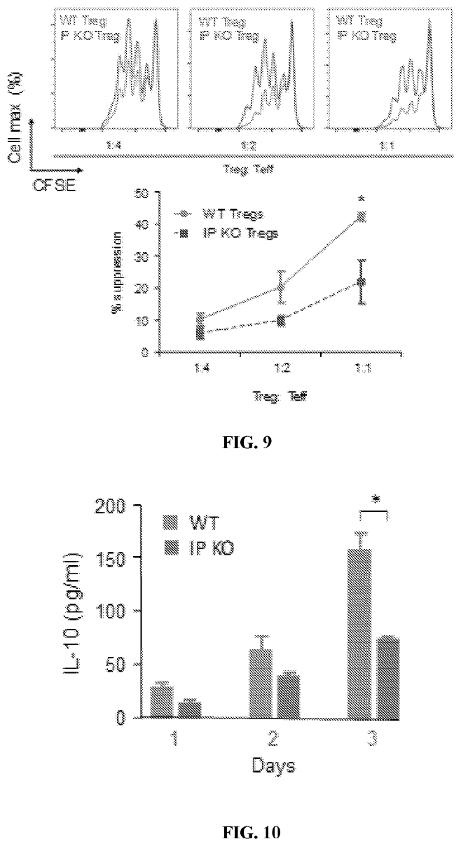

An investigation of whether PGI₂ orchestrates the function of Treg, which are important cellular mediators of tolerance was conducted. Tolerance is achieved when there are sufficient numbers of functioning Treg to suppress T effector cells (Teff). However, this balance can be altered by the presence of reduced numbers of Treg or less functional Treg, leading to inflammation with exposure to environmental antigens to which the host would ordinarily be tolerant. Given PGI₂'s role in maintaining tolerance, it was investigated whether PGI₂ creates a pro-tolerogenic environment by promoting Treg responses. In order to test this, Foxp3 GFP+ transgenic mice were obtained on a BALB/c background (Foxp3-GFP) from Jackson Labs (stock No. 006769) and then crossed these mice with IP KO mice to create IP KO mice in which Foxp3 cells express GFP (Foxp3-GFPxIP KO mice). Splenocytes were isolated from Foxp3-GFP and Foxp3-GFPxIP KO mice and isolated Treg that have the phenotype CD4+CD25+Foxp3+, to determine the per cell expression of Foxp3 in Treg from WT and IP KO mice. The master transcriptional regulator Foxp3 is critical for Treg development and function, and per cell expression of Foxp3, as determined by the mean fluorescence intensity of Foxp3, correlates with Treg functionality. The gating strategy to identify Foxp3-GFP expressing Treg is shown in FIG. 7. It was found that Treg from IP KO mice expressed significantly less Foxp3 on a per cell basis that WT Treg (FIG. 8). Given the decreased Foxp3 expression in the splenic Treg in IP KO mice, it was investigated whether IP KO Treg have decreased ability to suppress T effector cells Teff compared to WT Treg. To test this, splenic T cells were isolated from Foxp3-GFP and Foxp3-GFPxIP KO mice using antibodies conjugated to magnetic beads. Teff were enriched through negative selection in which remaining cells were CD4+CD25−. Subsequent positive selection was used to isolate CD4+CD25+Treg. Teff from WT mice were labeled with the fluorescent dye CFSE to assess proliferation and then stimulated with anti-CD3 and anti-CD28. These cells were then co-cultured with varying dilutions of Treg from either Foxp3-GFP or Foxp3-GFPxIP KO mice to determine the suppressive function of the Treg. After 3 days of culture, CFSE dilution was detected by flow cytometry as a metric of Teff proliferation. As cells undergo rounds of proliferation, the amount of CSFE in each of the progeny cells is half that of the parent cell, and each subsequent peak to the left representing the rounds of proliferation that the original parent Teff underwent. This assay allowed the determination of the suppressive function of Treg from WT mice and IP KO mice. As shown in FIG. 9, a significantly greater proportion of Teff co-cultured with Treg from IP KO mice, underwent proliferation compared to Teff that were co-cultured with WT Treg, shown in red at the ratio of 1 Treg:1 Teff. This indicates that Treg from IP KO mice were significantly less suppressive compared to Treg from WT mice.

To investigate what cellular-produced factor could account for the difference in function between Treg from IP KO and WT mice, Treg was also stimulated with anti-CD3 and anti-CD28 in the absence of Teff cells. It was found that at day 3 of culture that activated Treg from IP KO mice had decreased production of the immunoinhibitory cytokine IL-10 compared to Treg from WT mice (FIG. 10). It was also tested if the inability to signal through IP signaling inhibits inducible Treg (iTreg) differentiation from naïve CD4+ T cells. iTreg are generated from naïve CD4+ T cells in the periphery in response to immune insult or challenge and iTreg act synergistically with Treg to mediate tolerance. To test this, splenic T cells were isolated from WT and IP KO mice. CD4+ T cells were obtained using a negative selection step followed by a positive selection step to isolate naïve CD4+CD62L+ T cells. Naïve CD4+ T cells were cultured using IL-2 and TGF-β to induce iTreg differentiation, and iTreg differentiation was assessed by flow cytometry after 6 days of culture. It was found that under Treg-polarizing conditions, significantly fewer naïve CD4+ T cells from IP KO mice differentiated into iTreg, again defined as CD4+CD25+Foxp3+ cells. This additionally supports the concept that IP signaling is critical for Treg development. In summary, it was found that the inability to signal through IP decreased Treg Foxp3 expression, decreased Treg suppressive function, and decreased iTreg differentiation, showing that PGI₂ signaling through IP creates a protolerogenic environment that promotes Treg function and as a consequence, inhibits Teff responses. These results show that PGI₂ (or an analog) is a therapeutic strategy to increase Treg function in the setting of solid organ transplantation.

### Endogenous PGI₂ Signaling Effects on Heterotopic Heart Transplantation Survival

Initial work suggested that PGI₂ promotes immune tolerance by enhancing Treg function and inhibits T cell mediated solid organ rejection by restraining T effector cell function. However, PGI₂ has never been investigated in the immunobiology of alloreactivity and transplant rejection. Thus, endogenous PGI₂ signaling is analyzed for prolonging solid organ transplant survival. The heterotopic heart transplant model is an excellent model for acute T cell rejection and this system can also model acute and chronic antibody-mediated rejection. It is also the most common model to investigate immune tolerance. In this model, the heterotopic heart graft is transplanted into the peritoneal cavity of the recipient with an end-to-end anastomosis of the heart graft aorta to the recipient abdominal aorta and the graft pulmonary artery to the recipient inferior vena cava. Once the anastomosis is complete, the cardiac graft almost immediately starts contracting. The benefit of the cardiac transplantation model is the immediate vascularization and the ability to determine the graft function by palpating the graft's heartbeat. Complete MHC disparate cardiac allografts into immunocompetent hosts are usually rejected in 7-10 days and the pathology is similar to acute cell-mediated rejection in human transplantation.

In allogeneic models of cardiac transplantation, donor allografts lacking MHC class II or both class I and II had prolonged survival, whereas class I-deficient allografts (which did express allogeneic class II) were rapidly rejected. Similarly, heterotopic MHC mismatched hearts were rejected rapidly in CD8-deficient recipients, but were not rejected in CD4-deficient recipients. These studies reveal an essential interaction between MHC class II and CD4+ T cells for immune rejection of grafted hearts. The importance of the MHC class II-CD4+ T cell interaction in the mouse model of cardiac transplantation supports the contention that PGI₂ signaling protects against cardiac allograft rejection as it was shown that PGI₂ signaling inhibited prevented antigen-specific DC activation of CD4+ cells.

### Role of Endogenous PGI₂ Signaling in Cardiac Transplant Graft Survival

In this example, mice are investigated for the ability to signal through the PGI₂ receptor IP and have prolonged cardiac graft survival compared to mice that are IP-deficient. To test this, WT or IP KO mice are used on the C57BL/6 genetic background as recipients of WT BALB/c (H2^{d}) transplanted hearts. Both the WT and IP KO recipients are Foxp3-GFP reporter mice so that it is possible to enumerate Treg. In addition, WT C57BL/6 (H2^{b}) genetic background donors are used as a syngeneic control (group 3). Following transplantation, the heart function is monitored daily by palpation of the abdominal wall. Cessation of a palpable heart beat signals the end of graft function. Heterotopic WT mismatched cardiac allografts reject rapidly, usually within 7-10 days after transplantation. The impact of sex mismatch on transplant outcome remains debated, even though donor-recipient sex mismatch appears undesirable in female recipients. To address sex as a biological variable, both male and female mice are used, but only transplant organs of one sex into recipients of the same sex. To detect a statistically significant difference of 30% between groups 1 and 2, power analysis indicates that 8 mice per group are used.

### Role of Endogenous PGI₂ Signaling in Immune Cell Infiltration of Cardiac Allografts

Prior reports indicate that allospecific immune responses directed toward MHC mismatched solid organ transplants occur rapidly. It is next investigated whether IP-deficient recipient mice on a C57BL/6 genetic background that are transplanted BALB/c hearts have increased immune cell infiltration compared to WT C57BL/6 recipients. To test this, the grafts and immune responses are examined 5 days after transplantation, a time at which prior published studies have shown advanced cellular immune responses, while most allografts remain functional. Graft sections are stained with H&E to examine histopathology and cellular infiltrates. Experiments specifically focus on arterial vasculitis and venulitis; myocyte degradation; and cellular infiltrate in the interstitium, epicardium, and perivascular space. These are endpoints which have been shown to be present in an accelerated pace in cardiac transplants in which there are intact MHC II-CD4+ T cell interactions compared to transplants that lack competent MHC II-CD4+ signaling. Given that the WT and IP KO recipients are Foxp3-GFP reporter mice, immunofluorescence is performed to assess the number of Foxp3-GFP+ Treg to correlate this with graft survival.

### Effect of Exogenous PGI₂ Administration on Heterotopic Heart Transplantation Survival

Based on initial data that reveals that the PGI₂ analog cicaprost decreases T cell IL-2 production and NFATc activation, it is investigated whether exogenous PGI₂ analog administration prolongs solid organ transplant survival. Therefore, to test this, the PGI₂ analog iloprost is used, which is FDA approved for the treatment of pulmonary hypertension. For these experiments, iloprost is administered through a mini-osmotic pump system, starting the day prior to cardiac transplantation. The mini-osmotic pump system for drug delivery is used to achieve constant tissue levels of the PGI₂ analog. This device has been routinely used in the laboratory. Each of the mice in this experiment has an Alzet Micro-osmotic pump (2004) inserted through a small incision made through the skin between the scapulae of the anesthetized mice. The pump is easily inserted through the incision and the surgical site is closed with sutures. Prior to insertion, the pumps are handled and filled under aseptic conditions. This particular pump model (2004) has a reservoir of 200 μl with a delivery rate of 0.25 μl/hr. The duration of administration of the pharmacologic agent for this particular pump model at the delivery rate of 0.25 μl/hr is 28 days, approximately 2.5 weeks longer than anticipated time of histoincompatible transplant rejection published in the literature that is mentioned earlier. The identical system has been used to administer treprostinil successfully used in a rat model, with chronic administration of treprostinil decreasing the degree of experimentally-induced pulmonary hypertension. Iloprost is used in this example (rather than treprostinil) because it has greater specificity for IP than treprostinil, which also activated the PGE₂ receptor EP2. In these experiments, iloprost is initially administered at 10 ng/kg/min, the starting dose of iloprost used in the treatment of pulmonary hypertension.

### Role of Exogenous PGI₂ Analog Administration in Cardiac Transplant Graft Survival

In this example, it is investigated whether the administration of the PGI₂ analog iloprost prolongs cardiac graft survival compared vehicle treatment. To test this, two groups of WT C57BL/6 genetic background recipients of WT BALB/c transplanted hearts were used. The recipients are Foxp3-GFP reporter mice so that Treg can be enumerated and their function can be determined. Group 1 is treated with the vehicle for iloprost, methylacetate, via the Alzet miniosmotic pump, while group 2 is treated with iloprost as described above. The mini-osmotic pump containing either iloprost or its vehicle, methylacetate, is placed one day prior to transplant. The endpoints and the number of mice harvested for each endpoint here is the same as discussed above.

### Role of Exogenous PGI₂ Analog Administration in Immune Cell Infiltration of Cardiac Allografts

In this example, it is also investigated whether the administration of the PGI₂ analog iloprost decreases immune cell infiltration compared vehicle treatment. To test this, the same 2 groups of mice outlined above are used and the endpoints are identical to those above.

### Ability of an Exogenously Administered PGI₂ Analog to Modulate Treg Function

In this example, the effect of exogenously administered iloprost on Treg Foxp3 expression and suppressive function is investigated. To test this, spleens from the two groups of mice above are harvested and the Foxp3-GFP+ Treg are isolated by flow cytometry as described in the section above. Foxp3-GFP expression is then measured in both groups of mice and Treg suppression assays are also performed, as described above.

### Summary

In summary, PGI₂ (or a PGI₂ analog) administration can be used as a novel therapeutic strategy to prevent solid organ transplantation rejection. The data in this example shows that PGI₂ has many of the immunoinhibitory features of calcineurin inhibitors without the toxicity associated with those drugs. This technology advances the field of transplantation medicine, and provides a paradigm shift in identifying a novel therapeutic strategy to treat solid organ transplant recipients to decrease rejection of the incredibly valuable resource of human organs.

### REFERENCES CITED IN THIS EXAMPLE

1. Transplantation Immunology. In: Abbas A K, Lichtman A H, Pillai S, eds. Cellular and Molecular Immunology. Ninth edition ed. Philadelphia: Elsevier; 2018: 373-396.
2. Soderlund C, Radegran G. Immunosuppressive therapies after heart transplantation—The balance between under- and over-immunosuppression. Transplant. Rev. (Orlando.). 2015; 29(3):181-189.
3. Karam S, Wali R K. Current State of Immunosuppression: Past, Present, and Future. Crit Rev. Eukaryot. Gene Expr. 2015; 25(2):113-134.
4. Benghiat F S, Graca L, Braun M Y, et al. Critical influence of natural regulatory CD25+ T cells on the fate of allografts in the absence of immunosuppression. Transplantation. 2005; 79(6):648-654.
5. Karim M, Feng G, Wood K J, Bushell A R. CD25+CD4+ regulatory T cells generated by exposure to a model protein antigen prevent allograft rejection: antigen-specific reactivation in vivo is critical for bystander regulation. Blood. 2005; 105(12):4871-4877.
6. June B W, Felix N J, Griffiths R, et al. Prolonged survival of class II transactivator-deficient cardiac allografts. Transplantation. 2002; 74(9):1341-1348.
7. Zhou W, Hashimoto K, Goleniewska K, et al. Prostaglandin I2 analogs inhibit proinflammatory cytokine production and T cell stimulatory function of dendritic cells. J Immunol. 2007; 178(2):702-710.
8. Zhou W, Blackwell T S, Goleniewska K, et al. Prostaglandin I2 analogs inhibit Th1 and Th2 effector cytokine production by CD4 T cells. J Leukoc. Biol. 2007; 81(3):809-817.
9. Zhou W, Goleniewska K, Zhang J, et al. Cyclooxygenase inhibition abrogates aeroallergen-induced immune tolerance by suppressing prostaglandin I receptor signaling. J. Allergy Clin. Immunol. 2014; 134(3):698-705.
10. Moncada S, Gryglewski R, Bunting S, Vane J R. An enzyme isolated from arteries transforms prostaglandin endoperoxides to an unstable substance that inhibits platelet aggregation. Nature. 1976; 263(5579):663-665.
11. Del P R, Hernandez G, I, Escribano-Subias P. The prostacyclin pathway in pulmonary arterial hypertension: a clinical review. Expert. Rev. Respir. Med. 2017; 11(6):491-503.
12. Hill N S, Badesch D, Benza R L, et al. Perspectives on oral pulmonary hypertension therapies recently approved by the U.S. Food and Drug Administration. Ann. Am. Thorac. Soc. 2015; 12(2):269-273.
13. Dorris S L, Peebles R S, Jr. PGI(2) as a Regulator of Inflammatory Diseases. Mediators. Inflamm. 2012; 2012:926968.
14. Jaffar Z, Ferrini M E, Buford M C, Fitzgerald G A, Roberts K. Prostaglandin I2-IP signaling blocks allergic pulmonary inflammation by preventing recruitment of CD4+ Th2 cells into the airways in a mouse model of asthma. J Immunol. 2007; 179(9):6193-6203.
15. Nagao K, Tanaka H, Komai M, Masuda T, Narumiya S, Nagai H. Role of prostaglandin I2 in airway remodeling induced by repeated allergen challenge in mice. Am. J Respir. Cell Mol. Biol. 2003.
16. Takahashi Y, Tokuoka S, Masuda T, et al. Augmentation of allergic inflammation in prostanoid IP receptor deficient mice. Br. J Pharmacol. 2002; 137(3):315-322.
17. Zhou W, Zhang J, Goleniewska K, et al. Prostaglandin I2 Suppresses Proinflammatory Chemokine Expression, CD4 T Cell Activation, and STAT6-Independent Allergic Lung Inflammation. J. Immunol. 2016; 197(5):1577-1586.
18. Oga T, Matsuoka T, Yao C, et al. Prostaglandin F(2alpha) receptor signaling facilitates bleomycin-induced pulmonary fibrosis independently of transforming growth factor-beta. Nat. Med. 2009; 15(12):1426-1430.
19. Ostroukhova M, Seguin-Devaux C, Oriss T B, et al. Tolerance induced by inhaled antigen involves CD4(+) T cells expressing membrane-bound TGF-beta and FOXP3. J Clin Invest. 2004; 114(1):28-38.
20. Seymour B W, Gershwin L J, Coffman R L. Aerosol-induced immunoglobulin (Ig)-E unresponsiveness to ovalbumin does not require CD8+ or T cell receptor (TCR)-gamma/delta+ T cells or interferon (IFN)-gamma in a murine model of allergen sensitization. J. Exp. Med. 1998; 187(5):721-731.
21. Stampfli M R, Wiley R E, Neigh G S, et al. GM-CSF transgene expression in the airway allows aerosolized ovalbumin to induce allergic sensitization in mice. Journal of Clinical Investigation. 1998; 102(9):1704-1714.
22. Hon S, Nomura T, Sakaguchi S. Control of regulatory T cell development by the transcription factor Foxp3. Science. 2003; 299(5609):1057-1061.
23. Xia G, He J, Zhang Z, Leventhal J R. Targeting acute allograft rejection by immunotherapy with ex vivo-expanded natural CD4+CD25+ regulatory T cells. Transplantation. 2006; 82(12):1749-1755.
24. Wan Y Y, Flavell R A. Regulatory T-cell functions are subverted and converted owing to attenuated Foxp3 expression. Nature. 2007; 445(7129):766-770.
25. Chong A S, Alegre M L, Miller M L, Fairchild R L. Lessons and limits of mouse models. Cold Spring Harb Perspect Med. 2013; 3(12):a015495.
26. Benza R L, Passineau M J, Anderson P G, Barchue J P, George J F. The role of fibrinolytic genes and proteins in the development of allograft vascular disease. J Heart Lung Transplant. 2011; 30(8):935-944.
27. George J F, Gooden C W, Guo L, Kirklin J K. Role for CD4(+)CD25(+) T cells in inhibition of graft rejection by extracorporeal photopheresis. J Heart Lung Transplant. 2008; 27(6):616-622.
28. Campos L, Deli B C, Kern J H, et al. Survival of MHC deficient mouse heterotopic cardiac allografts and xenografts. Transplant Proc. 1995; 27(1):254-255.
29. Krieger N R, Yin D P, Fathman C G. CD4+ but not CD8+ cells are essential for allorejection. J Exp Med. 1996; 184(5):2013-2018.
30. Pietra B A, Wiseman A, Bolwerk A, Rizeq M, Gill R G. CD4 T cell-mediated cardiac allograft rejection requires donor but not host MHC class II. J Clin Invest. 2000; 106(8):1003-1010.
31. Puoti F, Ricci A, Nanni-Costa A, Ricciardi W, Malorni W, Ortona E. Organ transplantation and gender differences: a paradigmatic example of intertwining between biological and sociocultural determinants. Biol Sex Differ. 2016; 7:35.
32. Riella L V, Yang J, Chock S, et al. Jagged2-signaling promotes IL-6-dependent transplant rejection. Eur J Immunol. 2013; 43(6):1449-1458.
33. Yang J, Li X, Al-Lamki RS, et al. Smad-dependent and smad-independent induction of id1 by prostacyclin analogues inhibits proliferation of pulmonary artery smooth muscle cells in vitro and in vivo. Circ. Res. 2010; 107(2):252-262.
34. Aronoff D M, Peres C M, Serezani C H, et al. Synthetic Prostacyclin Analogs Differentially Regulate Macrophage Function via Distinct Analog-Receptor Binding Specificities. J Immunol. 2007; 178(3):1628-1634.

### Example 3

### Prostaglandin I₂ (PGI₂) Administration to Promote Tolerance and Inhibit Rejection in Mouse Models of MHC Mismatched Kidney Transplantation

Studies have shown that PGI₂ is an important inhibitor of inflammation. Specifically, PGI₂ reduces dendritic cell (DC) production of pro-inflammatory cytokines and chemokines, inhibits their ability to induce T cell antigen-specific proliferation and production of cytokines, and decreases their ability to uptake antigen. Furthermore, PGI₂ acts directly on T cells to inhibit both CD4+ Th1 and Th2 effector cytokine production. Together, these studies identify PGI₂ as an agent that promotes immune tolerance. Moreover, the vascular-protective capabilities of PGI₂ are well described; however, the capacity of PGI₂ to promote immune tolerance and inhibit rejection in models of organ transplantation is unknown. Thus, the ability of both endogenous, using PGI₂ receptor deficient mice (IP mice), and exogenous, through prophylactic treatment with PGI₂ analogs, PGI₂ to promote tolerance and inhibit rejection in mouse models of MHC mismatched kidney transplantation is investigated.

IP mice, on a C57BL/6 background, were bred 10 generations to a Balb/c background. To study the endogenous immune tolerance capabilities of PGI₂, a kidney is transplanted from 10-week old C57Bl/6J mice into either age-matched IP or Balb/c mice that have undergone bilateral nephrectomy. Balb/c that were transplanted a kidney from a Balb/c mouse are used as a negative control for rejection. To study the exogenous immune tolerance capabilities of PGI₂, a kidney is transplanted from 10-week old C57Bl/6J mice into age-matched Balb/c mice that have undergone bilateral nephrectomy; half of the mice are treated 2 days prior to transplantation and throughout the post-transplantation period with the PGI₂ analog treprostinil. Balb/c mice that have undergone bilateral nephrectomy but have been transplanted a kidney from a Balb/c mouse are again used as a negative control. Both groups of mice are followed for 12 weeks post transplantation. Weight, posture, activity and fur texture are monitored, as occurrence of graft-versus-host disease (GVHD) is remotely possible after kidney transplantation. The development of inflammation and edema within the graft is monitored throughout the study using functional magnetic resonance imaging (MRI) with diffusion-weighted imaging (DWI) and mapping of T2-relaxation time. Towards the end of the study, mice are retro-orbitally injected with FITC-labeled inulin. Blood samples are collected from the tail at 3, 7, 10, 15, 20, 40 and 60 min after injection. Amount of fluorescence is determined from the plasma and is used to calculate the glomerular filtration rate (GFR), a marker of renal function. Urine samples are collected weekly from the animals to assess injury of the transplanted kidney which is quantified by measuring urinary albumin, nephrin, IL-18, and neutrophil gelatinase-associated lipocalin (NGAL) via an enzyme-linked immunosorbent assay (ELISA), as well as through quantification of kidney injury molecule-1 (KIM-1) staining on formalin fixed, paraffin embedded sections of the transplanted kidney collected at the end of the study. Degree of renal fibrosis and levels of superoxide is determined using formalin fixed, paraffin embedded sections. Renal fibrosis is assessed by quantifying amounts of collagen after staining the sections with Masson's trichrome. Levels of superoxide are quantified after staining the sections with dihydroethidium (DHE). Degree of immune cell infiltration is quantified using flow cytometry or immunohistochemistry. Single cell suspensions of kidneys are prepared and stained for CD45 (total leukocytes), CD3 (total T cells), CD4, CD8, F4/80 (macrophage marker), CD19 (total B cells) and Fixable Live/Dead stain and analyzed on the flow cytometer. Formalin fixed, paraffin embedded sections are stained for CD45 (total leukocytes), CD3 (total T cells), CD4, CD8, F4/80 (macrophage marker), CD19 (total B cells) and DAPI (nuclei) and analyzed on a confocal microscope. Whole kidney mRNA is screened by RT-PCR for expression of pro-inflammatory cytokines including IFNγ, TNFα, IL6, and IL17A, to determine subsets of immune cells present.

### REFERENCES CITED IN THE EXAMPLE

1. Zhou, W., Goleniewska, K., Zhang, J., Dulek, D. E., Toki, S., Lotz, M. T., Newcomb, D. C., Boswell, M. G., Polosukhin, V. V., Milne, G. L., Wu, P., Moore, M. L., FitzGerald, G. A., & Peebles, R. S., Jr. 2014. Cyclooxygenase inhibition abrogates aeroallergen-induced immune tolerance by suppressing prostaglandin I2 receptor signaling. J Allergy Clin Immunol. 134(3): 698-705 e695.
2. Zhou, W., Hashimoto, K., Goleniewska, K., O'Neal, J. F., Ji, S., Blackwell, T. S., Fitzgerald, G. A., Egan, K. M., Geraci, M. W., & Peebles, R. S., Jr. 2007. Prostaglandin I2 analogs inhibit proinflammatory cytokine production and T cell stimulatory function of dendritic cells. J Immunol. 178(2): 702-710.
3. Arehart, E., Gleim, S., Kasza, Z., Fetalvero, K. M., Martin, K. A., & Hwa, J. 2007. Prostacyclin, atherothrombosis, and cardiovascular disease. Curr Med Chem. 14(20): 2161-2169.
4. Takahashi, Y., Tokuoka, S., Masuda, T., Hirano, Y., Nagao, M., Tanaka, H., Inagaki, N., Narumiya, S., & Nagai, H. 2002. Augmentation of allergic inflammation in prostanoid IP receptor deficient mice. Br J Pharmacol. 137(3): 315-322.
5. Toki, S., Goleniewska, K., Huckabee, M. M., Zhou, W., Newcomb, D. C., Fitzgerald, G. A., Lawson, W. E., & Peebles, R. S., Jr. 2013. PGI(2) signaling inhibits antigen uptake and increases migration of immature dendritic cells. J Leukoc Biol. 94(1): 77-88.
6. Zhou, W., Blackwell, T. S., Goleniewska, K., O'Neal, J. F., Fitzgerald, G. A., Lucitt, M., Breyer, R. M., & Peebles, R. S., Jr. 2007. Prostaglandin I2 analogs inhibit Th1 and Th2 effector cytokine production by CD4 T cells. J Leukoc Biol. 81(3): 809-817.
7. Kawabe, J., Ushikubi, F., & Hasebe, N. 2010. Prostacyclin in vascular diseases.—Recent insights and future perspectives. Circ J. 74(5): 836-843.
8. Kim, J. M., Kim, S. J., Joh, J. W., Kwon, C. H., Jang, K. T., An, J., Ki, C. S., Kang, E. S., Shin, M., Kim, B. N., & Lee, S. K. 2011. Graft-versus-host disease after kidney transplantation. J Korean Surg Soc. 80 Suppl 1: S36-39.
9. Chen, X., Meng, X., Xu, Y., Xie, H., Yin, S., Li, H., Wu, L., & Zheng, S. 2016. Cytokine and human leukocyte antigen (HLA) profile for graft-versus-host disease (GVHD) after organ transplantation. Eur J Med Res. 21(1): 38.
10. Hueper, K., Gutberlet, M., Brasen, J. H., Jang, M. S., Thorenz, A., Chen, R., Hertel, B., Barrmeyer, A., Schmidbauer, M., Meier, M., von Vietinghoff, S., Khalifa, A., Hartung, D., Haller, H., Wacker, F., Rong, S., & Gueler, F. 2016. Multiparametric Functional MRI: Non-Invasive Imaging of Inflammation and Edema Formation after Kidney Transplantation in Mice. PLoS One. 11(9): e0162705.
11. Hueper, K., Hensen, B., Gutberlet, M., Chen, R., Hartung, D., Barrmeyer, A., Meier, M., Li, W., Jang, M. S., Mengel, M., Wacker, F., Rong, S., & Gueler, F. 2016. Kidney Transplantation: Multiparametric Functional Magnetic Resonance Imaging for Assessment of Renal Allograft Pathophysiology in Mice. Invest Radiol. 51(1): 58-65.
12. Rieg, T. 2013. A High-throughput method for measurement of glomerular filtration rate in conscious mice. J Vis Exp(75): e50330.
13. Vaidya, V. S., Ferguson, M. A., & Bonventre, J. V. 2008. Biomarkers of acute kidney injury. Annu Rev Pharmacol Toxicol. 48: 463-493.
14. Kumar, A., Hammad, A., Sharma, A. K., Mc-Cardle, F., Rustom, R., & Christmas, S. E. 2015. Oxidative stress in kidney transplant biopsies. Exp Clin Transplant. 13 Suppl 1: 207-213.
15. Fonseca, I., Reguengo, H., Almeida, M., Dias, L., Martins, L. S., Pedroso, S., Santos, J., Lobato, L., Henriques, A. C., & Mendonca, D. 2014. Oxidative stress in kidney transplantation: malondialdehyde is an early predictive marker of graft dysfunction. Transplantation. 97(10): 1058-1065.

### Example 4

### Treprostinil for Innate Allergic Airway Inflammation

In the last several decades, the prevalence of allergic rhinitis, asthma, and atopic eczema increased markedly in developed countries. For instance, allergic disease has become one of the most common causes of chronic illness and affects 40-50 million Americans. Similar trends have begun to emerge in developing countries that have witnessed progressive westernization. Mounting evidence suggests that drugs inhibiting cyclooxygenase (COX) enzymes in the arachidonic acid metabolic pathway may be contributing to the increased allergy prevalence. Epidemiological studies reveal a correlation between frequent use of COX inhibiting medications and increased risk of developing allergic disorders and asthma. Very recently, a positive association was reported between the intake of non-aspirin nonsteroidal anti-inflammatory drugs (NSAIDs) and current asthma in adult survivors of childhood cancer. In addition, there was a higher prevalence of new-onset asthma in subjects who regularly used NSAIDs other than aspirin compared to nonusers. Animal studies confirmed that COX inhibition increased allergic sensitization; augmented allergic airway inflammation; and enhanced lung expression of IL-4, IL-5, and IL-13, cytokines expressed by CD4 T helper 2 (Th2) cells, supporting a role for the COX pathway in regulating allergic inflammation. IL-4 is the primary driver of Th2 differentiation and isotype switching to IgE. IL-5 induces eosinophil development, recruitment to the lung, and activation. IL-13 is a central mediator of allergic airway responsiveness (AR) and mucus expression. It was reported that the nonselective COX pharmacologic inhibitor indomethacin, as well as selective COX-1 and COX-2 inhibitors, significantly increased lung Th2 cytokine protein expression, allergic inflammation, and airway responsiveness (AR) to methacholine. This increased allergic airway inflammation induced by COX inhibition was independent of leukotrienes, IL-5 and IL-13, and occurred in the absence of STAT6, a critical transcription factor in the major pathway of Th2 differentiation. It was also reported that COX inhibition during allergic sensitization significantly enhanced both primary and long-term memory Th2 responses.

The current example is the first to examine how a COX product, prostaglandin (PG)I₂, modulates the early innate allergic immune response to antigens from Alternaria alternata. Alternaria alternata is a ubiquitous aeroallergen and hypersensitivity to this fungus has been linked to severe asthma exacerbations leading to respiratory arrest in young persons. In this example, the role of a PGI₂ analog, treprostinil, in regulating allergic inflammation in the recently developed Alternaria model of allergic airway inflammation is examined. In this model, mice are not infected with Alternaria, but are instead challenged with Alternaria extract (Alt Ex), a commercially available skin test reagent (Greer, Lenoir N.C.) which contains the fungal antigens that elicit IgE-mediated hypersensitivity reactions such as asthma exacerbations in persons who are sensitized to these microbial products.

In these experiments, PGI₂ analogs were used that have a much longer half-life of 20-30 minutes than the half-life of PGI₂, which is 60-90 seconds. Iloprost and cicaprost suppressed LPS-induced expression of CD86, CD40, and MHC class II molecules by DCs and inhibited the ability of DCs to stimulate antigen-specific CD4 T cell proliferation and production of the Th2 cytokines IL-5 and IL-13. It was also reported that PGI₂ analogs inhibited the ability of CD4 T cells activated in the Th2 polarizing conditions to express IL-4, IL-5, and IL-13 in vitro. These results suggested that exogenous PGI₂ decreases allergic inflammatory responses in vivo and this was seen in the mouse model when iloprost inhibited the maturation and migration of lung DCs to the mediastinal LNs, resulting in decreased induction of an allergen-specific Th2 response in these nodes. In another model, pretreatment of antigen-specific Th2 cells with a PGI₂ analog inhibited allergic inflammation upon subsequent airway antigen exposure.

In this example, it was investigated whether treprostinil is a negative regulator of the expression in response to Alternaria airway challenge and, as a result, inhibits the development and function of lung Group 2 innate lymphoid cells (ILC2). Treprostinil is currently approved by the Food and Drug Administration (FDA) for the treatment of pulmonary hypertension. These results are also used for other fungi, as well as to other common allergens such as dust mites and cockroach, that have high levels of protease activity similar to Alternaria alternata.

Ordinarily, IL-5 and IL-13 are referred to as Th2 cytokines when they are made by this subset of CD4 T cells; however, in this situation where they are made by lung ILC2 and not CD4 cells, these cytokines are referred to as “Th2 cell-type” cytokines. The lung ILC2 is an innate lymphoid subset that has a similar cytokine secretion profile to other innate lymphoid cells such as nuocytes. Lung ILC2 are lineage negative (Lin⁻) in that they do not express CD3ε, CD19, CD11b, Gr-1, NK1.1, or Ter119. There is some controversy about the surface expression of a variety of proteins including SCA-1, c-kit, ICOS, CD45, and ST2, largely because different groups have used different combinations of these molecules to phenotype ILC2. However, there seems to be complete consensus that ILC2 have surface expression of CD25 (IL-2Rα) and CD127 (IL-7Rα). Therefore, lung ILC2 is defined as Lin⁻CD25⁺CD127⁺. In vitro experiments revealed purified Lin⁻CD25⁺CD127⁺ cells stimulated with PMA and ionomycin expressed large quantities of IL-5 and IL-13, moderate amounts of IL-17A, and low amounts of IL-4 and IFN-γ. Gene profile analysis of resting lung ILC2 revealed that they expressed high amounts of Gata3, Cd69, and Il2ra, but low amounts of Notch and Rorc transcripts. These innate lymphoid cells depend on the transcriptional repressor Id2 for their development. In vivo experiments reveal that lung ILC2 have a critical role in rapid Th2 cell-type inflammation in response to protease exposure. Proteases are important constituents of many allergens.

For instance, the ability of the house dust mite to generate an allergic response is largely dependent on its protease activity. Proteases disrupt mucosal integrity by digesting cell adhesion molecules and also act on protease-activated receptors to activate epithelial cells. Therefore, a protease containing aeroallergen, Alternaria alternata has been chosen as a stimulus for allergic airway inflammation in this example.

Alternaria alternata contains a cysteine protease and potently induces human airway epithelial cells to express cytokines such as IL-33, TSLP, and IL-25 via its protease activity. The protease activity of airborne microbial allergens may promote IL-33, TSLP, and IL-25 expression in the airways, resulting in the development and exacerbation of Th2 inflammation, especially in allergic subjects who already have a predisposition for Th2 cytokines expression in the airways. In mice, intratracheal (IT) administration of Alt Ex induced lung IL-5 and IL-13 expression that occurred as early as 6 hours. This time frame strongly suggests that Alt Ex induced an innate Th2 cell-type cytokine response. Depletion of lung ILC2 virtually abolished Alt Ex-induced lung Th2 cell-type protein expression, revealing that these cells, and not mast cells, basophils, eosinophils, or T cells were the source of IL-5 and IL-13 in this model.

While ILC2 are constitutively present in the lung and can be activated after a single Alt Ex exposure, a model of 4 consecutive day Alt Ex administrations was further developed that: 1) continues to provide the opportunity to examine innate Th2 cell-type inflammatory response, as adaptive immunity is not developed after only 4 days of antigen exposure, and 2) allows for the determination of how PGI₂ regulates ILC2-driven airway responsiveness, as IL-13 expression is markedly increased compared to one time Alt Ex exposure. In this model, WT and IP KO mice IT were challenged with either PBS or Alt Ex at a concentration of 5 μg/100 μl PBS for either 1 day or 4 consecutive days and measured IL-13 protein in lung homogenates. Twenty-four hours after 1 day of Alt Ex challenge, there was a two-fold increase in lung IL-13 protein expression in the IP KO mice compared to WT mice (31.2±1.7 vs 13.4±2.7 pg/ml; n=3 per group). As shown in FIG. 11, 24 hours after the 4 days of IT Alt Ex challenge, there was a 3.6-fold increase in lung IL-13 expression in the IP KO mice compared to the WT (n=4 per group). While it is unlikely that the amount of lung IL-13 protein present following 1 day of IT Alt Ex challenge is sufficient to result in AR to methacholine, it is highly likely that the high level of IL-13 protein expression after 4 consecutive days of IT Alt Ex results in AR and this is assessed below.

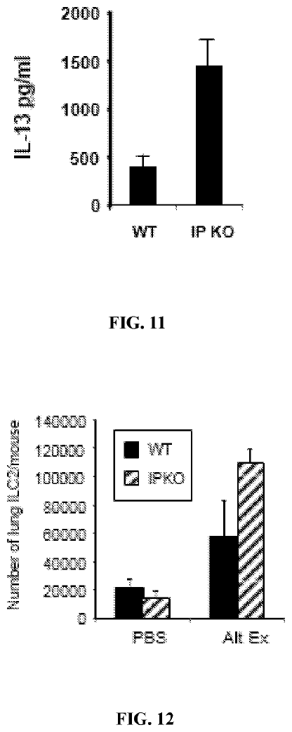

In another experiment using this four-day Alt Ex exposure model, lung ILC2 number was determined by flow cytometry for 3 mice in each group. ILC2 were defined as Lin⁻ CD25⁺CD127⁺ and the flow strategy is shown above after lymphoid cells were gated based on appropriate side and forward scatter characteristics. FIG. 12 reveals an almost 2-fold increase in the number of ILC2 cells of the IP KO mice challenged with 4 consecutive days of Alt Ex compared to the similarly treated WT mice, while there was no difference in the number of ILC2 between WT and IP KO mice that were challenged with PBS. When ANOVA was performed, this difference was not statistically significant because of the small numbers of mice in each group.

### Role of Treprostinil on Innate Allergic Airway Inflammation In Vivo

Data reveals that endogenous PGI₂ inhibited lung IL-5 protein expression 6 hours after IT Alt Ex administration. In this example, it is tested whether treprostinil regulates the innate Th2 cell-type cytokine response. It is important to understand the effects of treprostinil on the innate allergic immune response as such this agent is currently used therapeutically for pulmonary hypertension and can be used for allergic respiratory diseases such as asthma.

### Effect of Treprostinil on Innate Lung Th2 Cell-Type Inflammation In Vivo

As oral treprostinil is FDA approved for the treatment of pulmonary hypertension and therefore could possibly be a treatment for allergic respiratory diseases such as asthma, it is critical to determine if exogenous administration of this PGI₂ analog regulates the innate Th2 cell type immune response in vivo. Therefore, treprostinil inhibition of lung Th2 cell-type inflammation is investigated. To test this, 4 groups of mice are used with 35 mice in each group (Group 1: treatment=oral vehicle, challenge=Alt Ex; Group 2: treatment=oral treprostinil, challenge=Alt Ex; Group 3: treatment=SQ vehicle, challenge=Alt Ex; Group 4: treatment=SQ treprostinil, challenge=Alt Ex).

In this experiment, group 1 is treated with the vehicle for treprostinil by gavage starting five days prior to IT Alt Ex challenge. Group 2 is treated with treprostinil by gavage starting five days prior to IT Alt Ex challenge. Either the vehicle for treprostinil or treprostinil is started 5 days prior to IT Alt Ex challenge so that steady state could be reached prior to the IT Alt Ex challenge. In order to confirm that the gavage route of treprostinil administration is effective, experiments are performed in which treprostinil is administered by subcutaneously implanted mini-osmotic pump. The rationale for using oral administration is that patient compliance is greatest with an oral pharmacologic agent. The rationale for using the mini-osmotic pump system for drug delivery is to achieve constant tissue levels of the PGI₂ analog. Each of the mice in this experiment has an Alzet Micro-osmotic pump (1004) inserted through a small incision made through the skin between the scapulae of the anesthetized mice. The pump is easily inserted through the incision and the surgical site is closed with sutures. Prior to insertion, the pumps are handled and filled under aseptic conditions. This particular pump model (1004) has a reservoir of 100 μl with a delivery rate of 0.25 μl/hr. The duration of administration of the pharmacologic agent for this particular pump model at the delivery rate of 0.25 μl/hr is two weeks, approximately 6 days longer than the 2 days post challenge at when maximal AR is found, airway mucus expression, and peak lung IL-13 protein expression. The identical system has been used to administer treprostinil successfully in a rat model, with chronic administration of treprostinil decreasing the degree of experimentally-induced pulmonary hypertension. In this example, treprostinil is used because it has the greatest stability of any of the PGI₂ analogs currently available. In these experiments, treprostinil is initially administered at 1.25 ng/kg/min, the starting dose of treprostinil used in the treatment of pulmonary hypertension. The mini-osmotic pump containing either treprostinil or its vehicle and is be placed one day prior to infection. Based on data in which it was found that IP KO mice had greater airway TSLP protein expression compared to WT mice, power analysis reveals that the number of mice to detect a statistically significant 50% difference between the vehicle and treprostinil treated groups is 10 mice. In 10 mice, both lung and airway expression of IL-33 and IL-25 are measured, cytokines important in innate immune responses to allergen to determine whether treprostinil regulates Alt Ex-induced expression of these cytokines. Very little is known about PGI₂ regulation of chemokines. Therefore, the expression of epithelial-derived eosinophil chemotactic chemokines is examined including CCL3, CCL5, CCL7, CCL11, CCL13, CCL24, CCL26, as well as the Th2 promoting chemokines CCL17 and CCL22. The remaining five mice are harvested for histopathology and immunohistochemistry.

### Effect of Treprostinil on Innate Immunity-Driven Airway Responsiveness (AR)

Airway responsiveness (AR) as determined by methacholine reactivity is a defining criterion for the diagnosis of asthma in humans, with both sensitivity and specificity approaching 95%. A modification of this technique is also used in mice to define physiologic consequences of allergen challenge. There is a great deal of published experience using a system to measure AR in which mice are anesthetized, mechanically-ventilated, and administered methacholine through an internal jugular vein catheter. It is investigated whether treprostinil inhibits Alt Ex-induced AR. To test this, methacholine challenges are performed in the four groups of WT mice (above) treated with either vehicle or treprostinil.

In this experiment, methacholine challenge is performed on 10 mice from each group 24 hours after the last of 4 consecutive days of either PBS or IT Alt Ex administration for measurement of AR. It was previously found that this number of animals is necessary to detect significant difference in AR between groups that have a 2-3-fold difference in lung IL-13 expression. An additional 5 mice in each group are harvested for flow cytometry to enumerate lung ILC2 and to assess intracellular cytokine staining for IL-5 and IL-13 to confirm that lung ILC2 are the population expressing Th2 cell-type cytokines in this system. Ten mice are harvested 24 hours after the last Alt Ex administration to quantify airway eosinophilia and 5 mice are also harvested for histopathology to assess eosinophilia in the lung parenchyma and airway mucus expression by PAS staining.

### Statistical Analysis

For individual experiments, continuous variables (e.g., cytokine measurements and cell counts and differentials) are assessed using standard analysis of variance followed by post hoc tests such as the Sequential Neuman-Keuls test. Non-parametric tests (e.g., the Kruskal-Wallis test, a non-parametric analysis of variance; Wilcoxon Rank Sum test, a non-parametric between groups test) are also used to confirm the results of the primary analysis.

### REFERENCE CITED IN THIS EXAMPLE

(1) Eder W, Ege M J, von Mutius E. The asthma epidemic. N Engl J Med 2006; 355(21):2226-2235.
(2) Jarvis D, Burney P. ABC of allergies. The epidemiology of allergic disease. BMJ 1998; 316(7131):607-610.
(3) Asher M I, Montefort S, Bjorksten B et al. Worldwide time trends in the prevalence of symptoms of asthma, allergic rhinoconjunctivitis, and eczema in childhood: ISAAC Phases One and Three repeat multicountry cross-sectional surveys. Lancet 2006; 368(9537):733-743.
(4) Dunstan J A, Prescott S L. Does fish oil supplementation in pregnancy reduce the risk of allergic disease in infants? Curr Opin Allergy Clin Immunol 2005; 5(3):215-221.
(5) Prescott S L, Calder P C. N-3 polyunsaturated fatty acids and allergic disease. Curr Opin Clin Nutr Metab Care 2004; 7(2):123-129.
(6) Strachan D, Sibbald B, Weiland S et al. Worldwide variations in prevalence of symptoms of allergic rhinoconjunctivitis in children: the International Study of Asthma and Allergies in Childhood (ISAAC). Pediatr Allergy Immunol 1997; 8(4):161-176.
(7) Eneli I, Sadri K, Camargo C, Jr. et al. Acetaminophen and the risk of asthma: the epidemiologic and pathophysiologic evidence. Chest 2005; 127(2):604-612.
(8) Shaheen S O, Newson R B, Henderson A J et al. Prenatal paracetamol exposure and risk of asthma and elevated immunoglobulin E in childhood. Clin Exp Allergy 2005; 35(1):18-25.
(9) Marquis A, Strippoli M P, Spycher B D et al. Paracetamol, nonsteroidal anti-inflammatory drugs, and risk of asthma in adult survivors of childhood cancer. J Allergy Clin Immunol 2011; 127(1):270-272.
(10) Thomsen S F, Kyvik K O, Skadhauge L R et al. Regular use of non-steroidal anti-inflammatory drugs increases the risk of adult-onset asthma: a population-based follow-up study. Clin Respir J 2009; 3(2):82-84.
(11) Carey M A, Germolec D R, Bradbury J A et al. Accentuated T helper type 2 airway response after allergen challenge in cyclooxygenase-1−/− but not cyclooxygenase-2−/− mice. Am J Respir Crit Care Med 2003; 167(11):1509-1515.
(12) Gavett S H, Madison S L, Chulada P C et al. Allergic lung responses are increased in prostaglandin H synthase-deficient mice. Journal of Clinical Investigation 1999; 104(6):721-732.
(13) Jaffar Z, Wan K S, Roberts K. A key role for prostaglandin I2 in limiting lung mucosal Th2, but not Th1, responses to inhaled allergen. J Immunol 2002; 169(10):5997-6004.
(14) Laouini D, Elkhal A, Yalcindag A et al. COX-2 inhibition enhances the TH2 immune response to epicutaneous sensitization. J Allergy Clin Immunol 2005; 116(2):390-396.
(15) Abbas A K, Lichtman A H. Immediate Hypersensitivity—Chapter 19. In: Abbas A K, Lichtman A H, editors. Cellular and Molecular Immunology. Philadelphia: Elsevier Saunders, 2005: 432-452.
(16) Peebles Jr. R S, Dworski R, Collins R D et al. Cyclooxygenase inhibition increases interleukin 5 and interleukin 13 production and airway hyperresponsiveness in allergic mice. Am J Respir Crit Care Med 2000; 162:676-681.
(17) Peebles R S, Jr., Hashimoto K, Morrow J D et al. Selective cyclooxygenase-1 and -2 inhibitors each increase allergic inflammation and airway hyperresponsiveness in mice. Am J Respir Crit Care Med 2002; 165(8):1154-1160.
(18) Peebles R S, Jr., Hashimoto K, Sheller J R et al. Allergen-induced airway hyperresponsiveness mediated by cyclooxygenase inhibition is not dependent on 5-lipoxygenase or IL-5, but is IL-13 dependent. J Immunol 2005; 175(12):8253-8259.
(19) Hashimoto K, Sheller J R, Morrow J D et al. Cyclooxygenase inhibition augments allergic inflammation through CD4-dependent, STAT6-independent mechanisms. J Immunol 2005; 174(1):525-532.
(20) Zhou W, Newcomb D C, Moore M L et al. Cyclooxygenase inhibition during allergic sensitization increases STAT6-independent primary and memory Th2 responses. J Immunol 2008; 181(8):5360-5367.
(21) O'Hollaren M T, Yunginger J W, Offord K P et al. Exposure to an aeroallergen as a possible precipitating factor in respiratory arrest in young patients with asthma. N Engl J Med 1991; 324(6):359-363.
(22) Nagao K, Tanaka H, Komai M et al. Role of prostaglandin I2 in airway remodeling induced by repeated allergen challenge in mice. Am J Respir Cell Mol Biol 2003.
(23) Takahashi Y, Tokuoka S, Masuda T et al. Augmentation of allergic inflammation in prostanoid IP receptor deficient mice. Br J Pharmacol 2002; 137(3):315-322.
(24) Zhou W, Hashimoto K, Goleniewska K et al. Prostaglandin I2 analogs inhibit proinflammatory cytokine production and T cell stimulatory function of dendritic cells. J Immunol 2007; 178(2):702-710.
(25) Dorris S L, Peebles R S, Jr. PGI(2) as a Regulator of Inflammatory Diseases. Mediators Inflamm 2012; 2012:926968.
(26) Zhou W, Blackwell T S, Goleniewska K et al. Prostaglandin I2 analogs inhibit Th1 and Th2 effector cytokine production by CD4 T cells. J Leukoc Biol 2007; 81(3):809-817.
(27) Idzko M, Hammad H, van Nimwegen M et al. Inhaled iloprost suppresses the cardinal features of asthma via inhibition of airway dendritic cell function. J Clin Invest 2007; 117(2):464-472.
(28) Jaffar Z, Ferrini M E, Buford M C et al. Prostaglandin I2-IP signaling blocks allergic pulmonary inflammation by preventing recruitment of CD4+ Th2 cells into the airways in a mouse model of asthma. J Immunol 2007; 179(9):6193-6203.
(29) Neill D R, Wong S H, Bellosi A et al. Nuocytes represent a new innate effector leukocyte that mediates type-2 immunity. Nature 2010; 464(7293):1367-1370.
(30) Price A E, Liang H E, Sullivan B M et al. Systemically dispersed innate IL-13-expressing cells in type 2 immunity. Proc Natl Acad Sci USA 2010; 107(25):11489-11494.
(31) Barlow J L, Bellosi A, Hardman C S et al. Innate IL-13-producing nuocytes arise during allergic lung inflammation and contribute to airways hyperreactivity. J Allergy Clin Immunol 2012; 129(1):191-198.
(32) Doherty T A, Khorram N, Chang J E et al. STAT6 regulates natural helper cell proliferation during lung inflammation initiated by Alternaria. Am J Physiol Lung Cell Mol Physiol 2012; 303(7):L577-L588.
(33) Halim T Y, Krauss R H, Sun A C et al. Lung natural helper cells are a critical source of Th2 cell-type cytokines in protease allergen-induced airway inflammation. Immunity 2012; 36(3):451-463.
(34) Mjosberg J, Bernink J, Peters C et al. Transcriptional control of innate lymphoid cells. Eur J Immunol 2012; 42(8):1916-1923.
(35) Wolterink R G, Kleinjan A, van N M et al. Pulmonary innate lymphoid cells are major producers of IL-5 and IL-13 in murine models of allergic asthma. Eur J Immunol 2012; 42(5):1106-1116.
(36) Spits H, Di Santo J P. The expanding family of innate lymphoid cells: regulators and effectors of immunity and tissue remodeling. Nat Immunol 2011; 12(1):21-27.
(37) Yokota Y, Mansouri A, Mori S et al. Development of peripheral lymphoid organs and natural killer cells depends on the helix-loop-helix inhibitor Id2. Nature 1999; 397(6721):702-706.
(38) Phillips C, Coward W R, Pritchard D I et al. Basophils express a type 2 cytokine profile on exposure to proteases from helminths and house dust mites. J Leukoc Biol 2003; 73 (1):165-171.
(39) Kouzaki H, O'Grady S M, Lawrence C B et al. Proteases induce production of thymic stromal lymphopoietin by airway epithelial cells through protease-activated receptor-2. J Immunol 2009; 183(2):1427-1434.
(40) Kouzaki H, Iijima K, Kobayashi T et al. The danger signal, extracellular ATP, is a sensor for an airborne allergen and triggers IL-33 release and innate Th2-type responses. J Immunol 2011; 186(7):4375-4387.
(41) Axelgaard S, Holmboe S, Ringgaard S et al. Effects of chronic treprostinil treatment on experimental right heart hypertrophy and failure. Cardiol Young 2016; 1-11.
(42) Simonneau G, Barst R J, Galie N et al. Continuous subcutaneous infusion of treprostinil, a prostacyclin analogue, in patients with pulmonary arterial hypertension: a double-blind, randomized, placebo-controlled trial. Am J Respir Crit Care Med 2002; 165(6):800-804.
(43) Holgate S T. Innate and adaptive immune responses in asthma. Nat Med 2012; 18(5):673-683.
(44) Cockcroft D W. Direct challenge tests: Airway hyperresponsiveness in asthma: its measurement and clinical significance. Chest 2010; 138(2 Suppl):18S-24S.
(45) Hashimoto K, Durbin J E, Zhou W et al. Respiratory syncytial virus infection in the absence of STAT 1 results in airway dysfunction, airway mucus, and augmented IL-17 levels. J Allergy Clin Immunol 2005; 116(3):550-557.
(46) Newcomb D C, Boswell M G, Reiss S et al. IL-17A inhibits airway reactivity induced by respiratory syncytial virus infection during allergic airway inflammation. Thorax 2013.
(47) Peebles Jr. R S, Sheller J R, Johnson J E et al. Respiratory syncytial virus infection prolongs methacholine-induced airway hyperresponsiveness in ovalbumin sensitized mice. J Med Virol 1999; 57:186-192.
(48) Peebles R S, Jr., Sheller J R, Collins R D et al. Respiratory syncytial virus infection does not increase allergen-induced type 2 cytokine production, yet increases airway hyperresponsiveness in mice. J Med Virol 2001; 63(2):178-188.
(49) Peebles R S, Jr., Hashimoto K, Collins R D et al. Immune interaction between respiratory syncytial virus infection and allergen sensitization critically depends on timing of challenges. J Infect Dis 2001; 184(11):1374-1379.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1. A method of treating or preventing solid organ transplant rejection comprising: administering to a subject in need thereof prostacyclin or a prostacyclin analog; or a pharmaceutically acceptable salt thereof.

2. The method of claim 1, wherein the subject is administered prostacyclin.

3. The method of claim 2, wherein the prostacyclin is epoprostenol.

4. The method of claim 1, wherein the subject is administered a prostacyclin analog.

5. The method of claim 4, wherein the prostacyclin analog is selected from iloprost, cicaprost, treprostinil, or beraprost.

6. The method of claim 5, wherein the prostacyclin analog is iloprost.

7. The method of claim 1, wherein the solid organ transplant is a heart, kidney, lung, liver, or pancreas transplant.

8. The method of claim 7, wherein the solid organ transplant is a heart transplant.

9. The method of claim 7, wherein the solid organ transplant is a kidney transplant.

10. The method of claim 1, wherein the prostacyclin or a prostacyclin analog is administered by a method selected from the group consisting of intravenous, oral, and aerosol.

11. A method of treating or preventing allergic disease comprising: administering to a subject in need thereof prostacyclin or a prostacyclin analog; or a pharmaceutically acceptable salt thereof.

12. The method of claim 11, wherein the subject is administered prostacyclin.

13. The method of claim 12, wherein the prostacyclin is epoprostenol.

14. The method of claim 11, wherein the subject is administered a prostacyclin analog.

15. The method of claim 14, wherein the prostacyclin analog is selected from iloprost, cicaprost, treprostinil, or beraprost.

16. The method of claim 15, wherein the prostacyclin analog is iloprost.

17. The method of claim 11, wherein the allergic disease is selected from the group consisting of asthma, allergic rhinitis, allergic sinusitis, anaphylactic syndrome, urticaria, angioedema, atopic dermatitis, allergic contact dermatitis, erythema nodosum, and erythema multiforme.

18. The method of claim 17, wherein the allergic disease is asthma.

19. The method of claim 11, wherein the prostacyclin or a prostacyclin analog is administered by a method selected from the group consisting of intravenous, oral, and aerosol.

20. The method of claim 11, wherein the administration of the prostacyclin or the prostacyclin analog decreases IL33 expression.
